(19) United States
(12) Patent Application Publication
(10) Pub. No.: US 2012/0172375 A1
(43) Pub. Date: Jul. 5, 2012

(54) COMPOUNDS AND METHODS OF PROMOTING OLIGODENDROCYTE PRECURSOR DIFFERENTIATION

(76) Inventors: Bruce Trapp, Bentleyville, OH (US); Wendy B. MacKlin, Denver, CO (US); Carlos E. Pedraza, Arlington, MA (US); Elena K. Muratova, St. Petersburg (RU); Victor V. Sokolov, St. Petersburg (RU)

(21) Appl. No.: 13/128,124

(22) PCT Filed: Nov. 9, 2009
(86) PCT No.: PCT/US2009/063720

§ 371 Date: Nov. 3, 2011

Related U.S. Application Data
(60) Provisional application No. 61/112,449, filed on Nov. 7, 2008.
(60) Provisional application No. 61/149,929, filed on Feb. 4, 2009.

(51) Int. Cl. A61K 31/4965; A61P 25/00; C12N 5/02

(52) U.S. Cl. 514/255.01; 435/377

(57) ABSTRACT

A method of promoting the differentiation of an oligodendrocyte precursor cell. The method includes administering to the oligodendrocyte precursor cell an effective amount of a compound capable of promoting oligodendrocyte precursor cell differentiation.

### RELATED BACKGROUND

This application claims priority from U.S. Provisional Application Nos. 61/112,449, filed Nov. 7, 2008, and 61/149,929, filed Feb. 4, 2009, the subject matter, which is incorporated herein by reference.

### BACKGROUND

Multiple sclerosis (MS) is a complex neurological disease characterized by deterioration of central nervous system (CNS) myelin. This insulating material, composed in its majority by lipids (70% lipids, 30% protein), protects axons and makes possible the saltatory conduction, which speeds axonal electric impulse. Demyelination of axons in chronic MS may result in axon degeneration and neuronal cell death, but more specifically, MS destroys oligodendrocytes, the highly specialized CNS cells that generate and maintain myelin.

Oligodendrocyte precursors (PDGFRα+, NO2-proteoglycan+), the immature oligodendrocytes, are generated in ventral areas of the developing brain from a common glial progenitor, actively migrate and proliferate populating the CNS W finally differentiate to premyelinating oligodendrocytes (O4+). At this maturation point., oligodendrocytes both target and extend myelin sheaths along axons or they die. However, a population of oligodendrocyte precursors remains as resident, undifferentiated cells throughout their life supposedly to play a role as myelin recovering cells in damage or deterioration settings. Indeed, remyelination of early MS onset lesions has been reported which correlates with NG2+ oligodendrocyte progenitors detected in or around MS lesions. Nevertheless, complete, functional remyelination of MS lesions is not accomplished indicating a lack of effective maturation of resident oligodendrocyte precursors.

Less explored has been however, the hypothesis of remyelination by either endogenous oligodendrocyte precursors or transplanted cells. Transplantation of precursor cells from diverse sources has shown promising results in terms of survival and migration of exogenous cells for long distances. Remyelination to some extent has also been reported in several experimental models of demyelination after transplantation of neural precursors and stem cells. Yet, remyelination of multiple demyelinated areas by transplanted cells would require multiple transplantation loci, which in practice limits the effectiveness and clinical applicability of this approach.

Promoting remyelination by inducing differentiation of endogenous oligodendrocyte progenitors can stimulate and enhance intrinsic, natural remyelination. Therefore, there is a need for compounds and therapeutic methods capable inducing endogenous oligodendrocyte precursor differentiation.

### SUMMARY OF THE INVENTION

The present invention relates generally to compounds and methods for oligodendrocyte precursor cell differentiation. The present invention also relates to methods for the treatment of disease in subjects where remyelination by the induction of endogenous oligodendrocyte precursor differentiation is beneficial to the subject.

The present invention relates to a method of promoting oligodendrocyte precursor cell differentiation. The method includes administering to one or more oligodendrocyte precursor cells an effective amount of a compound selected from the following general structures:

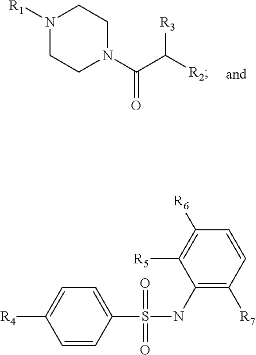

wherein R₁, R₂, R₃, R₄ R₅, and R₇ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, pharmaceutically acceptable salts thereof, and combinations thereof.

The present invention also relates to method of promoting oligodendrocyte precursor cell differentiation comprising administering to one or more oligodendrocyte precursor cells an effective amount of a compound selected from the group consisting of:

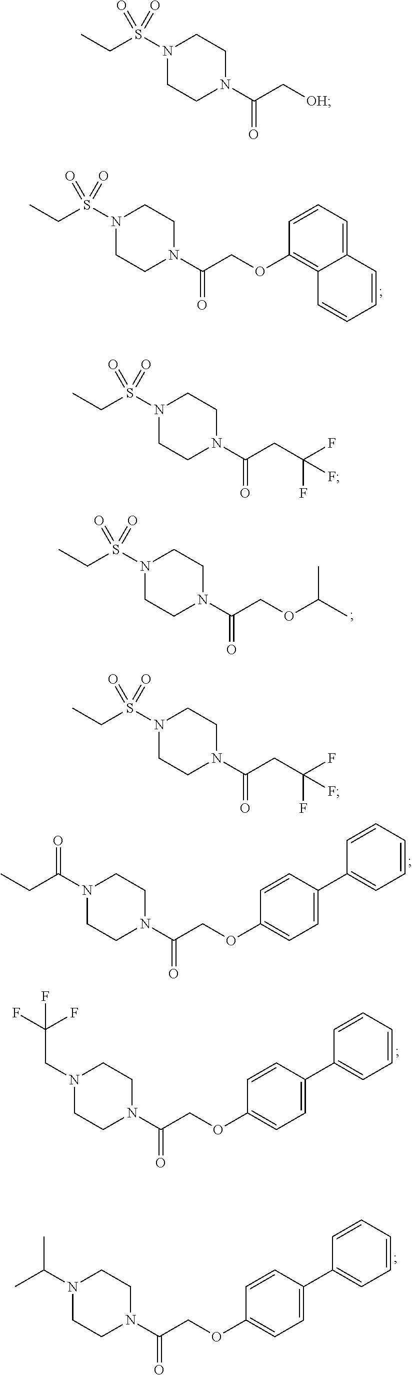

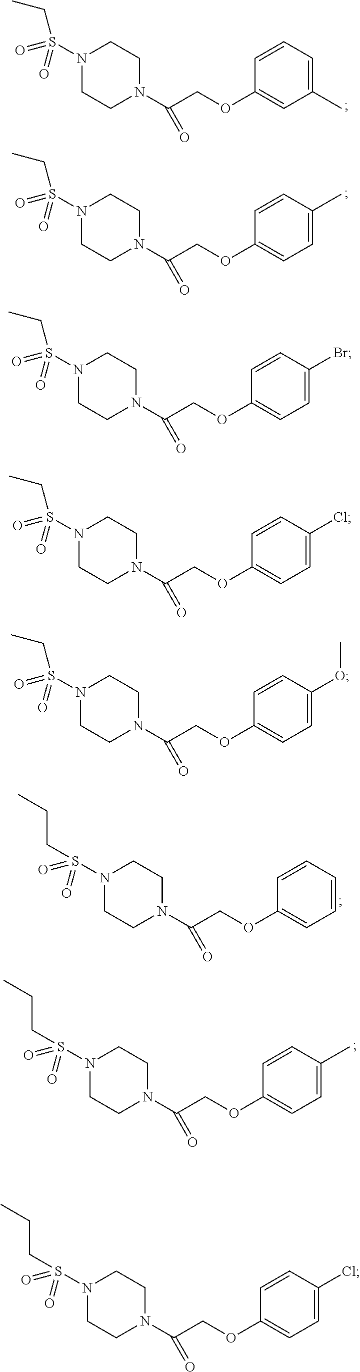

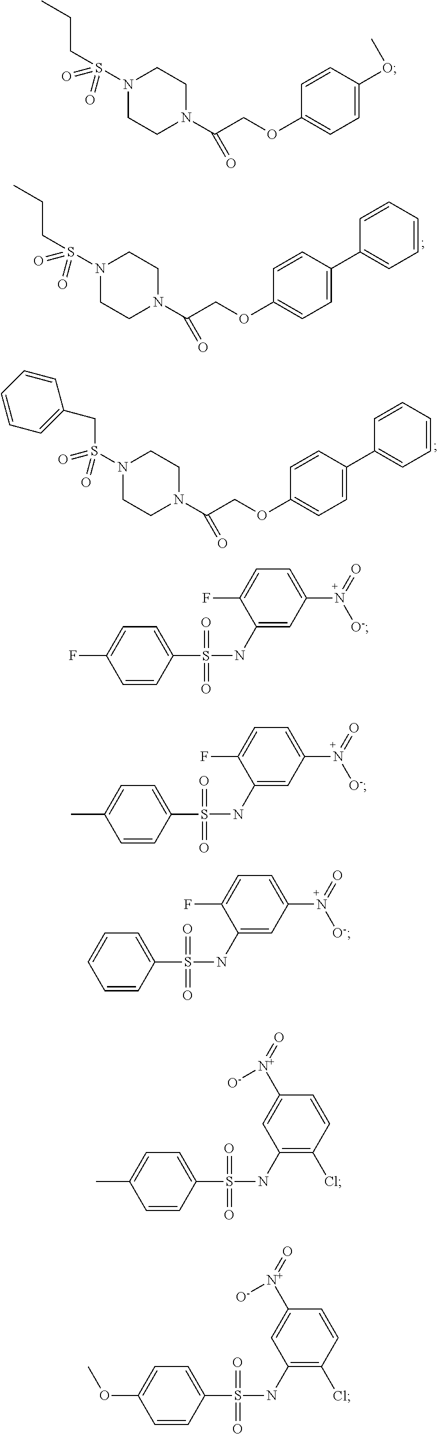

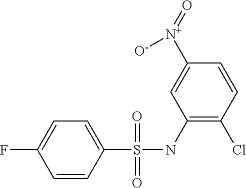

and pharmaceutically acceptable salts thereof.

The present invention further relates to a method of promoting oligodendrocyte precursor cell differentiation in a subject. The method includes administering to the subject a therapeutically effective amount of a compound selected from the following general structures:

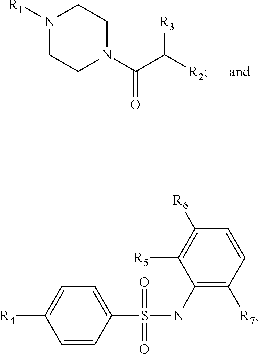

wherein R₁₁, R₂, R₃, R₄, R₅, R₆, and R₇, each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, pharmaceutically acceptable salts thereof, and combinations thereof.

The present invention also relates to a method of treating a neurodegenerative disease in a subject. The method includes administering to the subject a therapeutically effective amount of a compound selected from the following general structures:

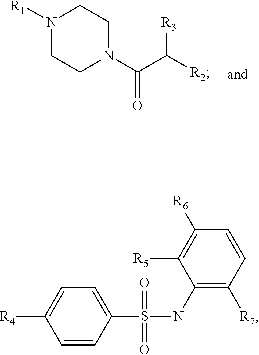

wherein R₁, R₂, R₃, R₄, R₅, R₆, and R₇, each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, carboxylato, carbamoyl, an amino, a substituted amino, an alkylamido, an arylamido, an imino, an alkylimino, an arylimino, a nitro, a nitroso, pharmaceutically acceptable salts thereof and combinations thereof.

The present inventing farther relates to a method of treating multiple sclerosis in a subject. The method includes administering, to the subject a therapeutically effective amount of a compound selected from the following general structures:

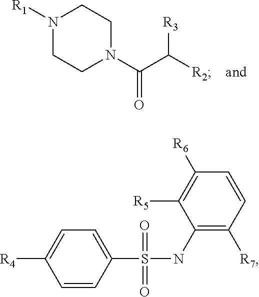

wherein R₁, R₂, R₃, R₄, R₅, R₆, and R₇ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkyloarbonato, an arylcarbonato, a carboxy, a carboxylato, a carbamoyl, an amino, a substituted amino, an alkylamido, an arylamido, an imino, an alkylimino, an arylimino, a nitro, a nitroso, pharmaceutically acceptable salts thereof, and combinations thereof.

The present invention also relates to a method of treating a neurodegenerative disease in a subject. The method includes the step of administering to the subject a therapeutically effective amount of a compound selected from the following general structures:

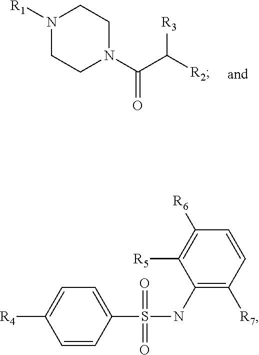

wherein R₁, R₂, R₃, R₄, R₅, R₆, and R₇, each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbony, an aryloxycarbonyl, halocarbonyl, an alkylcarbonato, an alylcarbonato, a carboxy, a carboxylato, a carbamoyl, an amino, a substituted amino, an alkylamido, an arylamido, an imino, an alkylimino, an arylimino, a nitro, a nitroso, salts thereof, and combinations thereof; and administering an additional anti-neurodegenerative disease agent to the subject.

### DESCRIPTION OF DRAWINGS

FIG. 1 illustrates (A) photographs and (B) an immunoassay showing oligodendrocyte precursor differentiation induced by small molecules. A. Oligodendrocyte progenitors were exposed for 48 hr to small molecule A (C20H24N2O4S, 10 μM) or the same volume of DMSO (Control). Note the evident morphology transformation from bipolar, elongated cells in the control (scores 1-2 on the differentiation scale), to multi-process, branched cells showing membranous structures in response to the small molecule treatment (score 4). Upper panels show phase contrast images of cells treated or not (control) with compound A. Immunostaining for PIP (Green) and MBP (Red) showed dramatic :morphological changes and increased expression of these myelin proteins h cells exposed to compound A. B. Effect on PLP/DM20 and CNPase expression of the thirteen positive compounds selected for the induction of oligodendrocyte progenitor differentiation.

FIG. 2 illustrates immunoassays and histograms showing the differentiation effect of compounds A. A-7, and B on oligodendrocyte precursors is dose and time dependent. Dose-dependency was determined by western blot for PLP/DM20 and CNPase a cells exposed to the indicated concentrations of compounds A, A-7 or B during 48 hrs. Histograms below western blot images show densitometry quantification of the bands normalized first to an internal control (β-tubulin of β-Actin) and her expressed relative to control levels. Induction of the expression of these myelin proteins was observed a low concentrations (0.01 and 0.1) for the three compounds (A). Cell differentiation was consistent with the increased expression of myelin proteins. Seven days after treatment with compounds A, A-7 and B, the cells showed formation of long-thick processes and major membranous structures characteristic of premyelinating oligodendrocytes. Quantification of mature cells was performed in two independent experiments and is presented as percentage of total cells in at least 5 fields totaling approximately 500 cells per condition (histogram). Results are average ±SD. (**p<0.01, student's t-test).

FIG. 3 illustrates photographs showing remyelination induced b small molecules in lysolecithin-demyelinated cerebellar slices. Cerebellar explants (10 days in vitro, div) were demyelinated by exposure to lysolecithin and subsequently treated with compounds A, A-7 or B for 10 additional days. Extensive populations of Plp-EGFP cells (left column, top panel) and myelin formation, as determined by immunostaining for non-phosphorilated neurofilament (red) and myelin basic protein (green, left column bottom panels), were observed in non demyelinated sections at the end of the experiment (21 div), In tissue slices demyelinated at 10 div and treated with vehicle, substantially reduced cell numbers, very little myelin and nude axons were observed (control column). On the other hand, evident oligodendrocyte cell recovery and myelin regeneration was observed around neurofilament-positive axons in explants treated with compounds A, A-7 or B. Note the immunostaining of myelin surrounding axons in explants treated with small molecules.

FIG. 4 illustrates photographs and a histogram showing treatment of zebrafish embryos with compound A and A-7 resulted m enhanced accumulation of oligodendrocyte progenitor proliferation and differentiation. tg[Plp:EGFP] embryos (6 hrs post-fertilization, bpi) were exposed to 10 nM compound A for 66 hrs. Embryos age at the end of the assay was 72 hpf. The top image shows a transgenic zebrafish and the demarked area represents the section of the spinal cord actually analyzed (Bar 200 μm). Confocal microscopy was used for high power images of treated or untreated fish. Note the increased accumulation of oligodendrocytes and enhanced differentiation induced by exposure of fish to compounds A and A-7 (Bar 25 μm).

FIG. 5 illustrates photographs and a histogram showing compounds A and A-7 enhance remyelination cuprizone-induced demyelination in mice. Strong demyelination induced by cuprizone-supplemented food was observed primarily in the caudal corpus callosum of C57B1/6J mice after 6 weeks on this diet. At this point, the mice were injected i.p. with either vehicle (DMSO) or the indicated doses of compounds A, A-7 (images) and B (histogram.) maintaining the mice in cuprizone one diet for 72 hrs. A second dose was then administered and the food was changed to regular chow to allow for natural myelin recovery for additional 48 hrs. Tissue sections were analyzed for remyelination by means of black and gold staining for total myelin. Quantification of the demyelinated area was performed b Image J software First normalizing the images to the lowest background possible and then scoring densitometry values above that threshold (**: p<0.01, Student's t-test),

FIG. 6 illustrates histograms showing recovery of myelin protein expression m demyelinated tissue from Mice injected with compounds A and A-7. Assessment of myelin protein re-expression, as a further indication of remyelination in animals injected with small molecules, was performed in tissue sections (30 μm) with anti-PLP and anti-MOG antibodies. Quantification in all conditions was performed with the Odyssey infrared scanner after immunostaining using infrared IRdye secondary antibodies (A and B). Quantification is represented as percentage of no cuprizone and was performed in four animals per condition (**: p<0.01, Student's t-test). The content of oligodendrocyte progenitor cells positive for Platelet-Derived Growth Factor Receptor-alpha (PDGFRα) were counted in the ventral corpus callosum area in a 25 μm thick confocal microscope Z-series (C).

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

In the present specification, the structural formula of the compound represents a certain isomer for convenience in some cases, but the present invention includes all isomers such as geometrical isomer, optical isomer based on an asymmetrical carbon, stereoisomer, tautomer and the like which occur structurally and an isomer mixture and is not limited to the description of the formula for convenience, and may be any one of isomer or a mixture. Therefore, an asymmetrical carbon atom may be present in the molecule and an optically active compound and a racemic compound may be present in the present compound, but the present invention is not limited to them and includes any one. In addition, a crystal polymorphism may be present but is not limiting, but any crystal form may be single or a crystal form mixture, or an anhydride or hydrate. Further, so-called metabolite, which is produced by degradation of the present compound in vivo, is included in the scope of the present invention.

It will be noted that the structure of some of the compounds of the invention include asymmetric (chiral) carbon atoms. It is to be understood accordingly that the isomers arising from such asymmetry are included within the scope of the invention, unless indicated otherwise. Such isomers can be obtained in substantially pure form by classical separation techniques and by stereochemically controlled synthesis. The compounds of this invention may exist in stereoisomeric form, therefore can be produced as individual stereoisomers or as mixtures.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

Chiral isomer means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et at, Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J., Chem. Educ. 1964, 41, 116).

“Geometric Isomers” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture however, as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and Recrystallization solvent, rate of crystallization, storage temperature, and other factors may cause one crystal form to dominate. Crystal polymorphs of the compounds can be prepared by crystallization under different conditions.

Additionally, the compounds of the present invention, for example, the salts of the compounds, can exist in either hydrated or unhydrated the anhydrous) form or as solvates with other solvent molecules. Nonlimiting examples of hydrates include monohydrates, dihydrates, etc. Nonlimiting examples of solvates include ethanol solvates, acetone solvates, etc.

“Solvates” means solvent addition forms that contain either stoichiometric or non stoichiometric amounts of solvent. Some compounds have a tendency to trap a fixed molar ratio of solvent molecules m the crystalline solid state, thus forming a solvate. If the solvent is water the solvate formed is a hydrate, when the solvent is alcohol, the solvate formed is an alcoholate. Hydrates are formed by the combination or one or more molecules of water with one of the substances in which the water retains its molecular state as H₂O, such combination being able to form one or more hydrate.

“Tautomers” refers to compounds whose structures differ markedly in arrangement of atoms, but which exist in easy and rapid equilibrium. It is the understood that compounds of Formula I may be depicted as different tautomers. It should also be understood that when compounds have tautomeric forms, all tautomeric forms are intended to be within the scope of the invention, and the naming of the compounds does not exclude any tautomer form.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative”, refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

The phrases “parenteral administration” and “administered parenterally” are art-recognized terms, and include modes of administration other than emend and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject which may be predisposed to the disease, disorder and/or condition but has not yet been diagnosed as having it. Preventing a condition related to a disease includes stopping the condition from occurring after the disease has been diagnosed but before the condition has been diagnosed.

A “pharmaceutical composition” is a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to he particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of a compound of this invention include powders, sprays, ointments, pastes, creams, lotions, gels, solutions, patches and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” is art-recognized. In certain embodiments, the term includes compositions, polymers and other materials and/or dosage forms which are within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which /nay serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose, acetate; (4) powdered tragacanth; (5) malt; (6) gelatin; (7) talc; (8) excipients, such as cocoa butter and suppository, waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compounds of the invention are capable of further forming salts. All of these forms are also contemplated within the scope of the claimed invention.

“Pharmaceutically acceptable salt” of a compound means a salt that is pharmaceutically acceptable and that possesses the desired pharmacological activity of the parent compound.

For example, the salt can be an acid addition salt. One embodiment of an acid addition salt is a hydrochloride salt

The pharmaceutically acceptable salts of the present invention can h synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two generally, non-aqueous media like ether, ethyl acetate, ethanol, isopropanol, or acetonitrile are preferred. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl mine-containing, and imine-containing compounds of the present invention.

It should be understood that all references to pharmaceutically acceptable salts include solvent addition forms (solvates) or crystal forms (polymorphs) as defined herein, of the same salt.

The compounds of the present invention can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds of the present invention can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy, or carbonyl group is bonded to any group that may be cleaved in vivo to form a free hydroxyl, free amino, free sulftydryl, free carboxy or free carbonyl group, respectively.

Examples of prodrugs include, but are not limited to esters (e.g., acetate, dialkylaminoacetates, formates, phosphates, sulfates, and benzoate derivatives) and carbamates N,N-dimethylaminocarbonyl) of hydroxy functional groups, ester groups (e.g. ethyl esters, morpholinoethanol esters) of carboxyl functional groups, N-acyl derivatives (e.g. N-acetyl) N-Mannich bases, Schiff bases and enaminones of amino functional groups, oximes, acetals, ketals and enol esters of ketone and aldehyde functional groups in compounds of Formula I, and the like, See Bundegaard, H. “Design of Prodrugs” p1-92, Elesevier, New York-Oxford (1985).

“Protecting group” refers to a grouping of atoms that when attached to a reactive group a molecule, masks, reduces or prevents that reactivity. Examples of protecting groups can be found in Green and Wilts, Protective Groups in Organic Chemistry, (Wiley, 2.sup.nd ed. 1991); Harrison and Harrison et al., Compendium of Synthetic Organic Methods, Vols. 1-8 (John Wiley and Sons, 1971-1996); and Kocienski, Protecting Groups, (Verlag, 3.sup.rd ed. 2083).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition, such as macular degeneration or other forms of retinal disease whose etiology involves aberrant clearance of all trans-retinal. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “effective amount” are art-recognized terms. In certain embodiments, “therapeutically effective amount” or “effective amount”, in terms of each foregoing methods, is the amount of the compounds described herein effective to induce or promote differentiation of at least one oligodendrocyte precursor.

The term “ED50” is art-recognized. In certain embodiments. ED50 means the dose of a drug, which produces 50% of its maximum response or effect, or alternatively, the dose which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

With respect to any chemical compounds, the present invention is intended to include all isotopes it atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The chemical compounds described herein can have asymmetric centers. Compounds of the present invention containing an asymmetrically substituted atom can be isolated in optically active or racemic forms. It is well known in the art how to prepare optically alive forms, such as by resolution of racemic firms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described arid can be isolated as a mixture of isomers or as separated isomeric forms. All chiral diastereomeric racemic, and geometric isomeric forms of a structure are intended, unless the specific stereochemistry or isomeric form is specifically indicated. All processes used to prepare compounds of the present invention and intermediates made therein are, where appropriate, considered to be part of the present invention. All tautomers of shown or described compounds are also, where appropriate, considered to be part of the present invention.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2 2-6, 2-5, 2-4, 2-3, 3-6, 3-5, 3-4, 4-6, 4-5, and 5-6 carbons,

The phrase “having the formula” or “having the structure” is not intended to be limiting and is used in the same way that the term “comprising” is commonly used.

“Effective amount”, in terms of each foregoing methods, is the amount of the compounds described herein effective to induce or promote differentiation of at least one oligodendrocyte precursor.

The term “alkyl” refers to a branched or unbranched saturated hydrocarbon group typically although not necessarily containing 1 to about 24 carbon atoms, such as methyl, ethyl, n-propyl isopropyl, i-butyl, isobutyl, t-butyl, octyl, decyl, and the like, as well as cycloalkyl groups, such as cyclopentyl, cyclohexyl, and the like. Generally, although again not necessarily, alkyl groups herein contain 1 to about 18 carbon atoms, preferably 1 to about 12 carbon atoms.

“Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the term “alkyl” includes linear, branched, cyclic, unsubstituted, substituted, and/or heteroatom-containing alkyl, respectively.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, cyclopentenyl, cyclohexenyl, cyclooctenyl, and the like. Generally, although again not necessarily, alkenyl groups car, contain 2 to about 18 carbon atoms, and more particularly 2 to 12 carbon atoms. The term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl or heterocycloalkenyl (e.g., heterocylcohexenyl) in which at least one carbon atom is replaced with a heteroatom. If not otherwise indicated, the terms “alkenyl” includes linear, branched, cyclic, unsubstituted, substituted, and/or heteroatom-containing alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily alkynyl groups can contain 2 to about 18 carbon atoms, and more particularly can contain 2 to 12 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If-not otherwise indicated, the term “alkynyl” include linear, branched unsubstituted, substituted, and/or heteroatom-containing alkynyl, respectively.

The term “alkoxy” refers to an alkyl group hound through a single, terminal ether linkage; that is, an “alkoxy” group may be represented as -0-alkyl where alkyl is as defined above, A “lower alkoxy” group intends an alkoxy group containing 1 to 6 carbon atoms, and includes, for example, methoxy, ethoxy, propoxy, isopropoxy, t-butyloxy, etc.

The term “aryl” refers to an aromatic substituent containing a single aromatic ring or multiple aromatic rings that are fused together directly linked, or indirectly linked (such that the different aromatic rings are bound to a common group such as a methylene or ethylene moiety). Aryl groups can contain 5 to 20 carbon atoms, and, for example, can contain 5 to 14 carbon atoms. Examples aryl groups contain one aromatic ring or two used or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatics.

The term “aryloxy” as used herein refers to an aryl group bound through a single, terminal ether linkage, wherein “aryl” is as deface above. An “aryloxy” group may be represented as —O-aryl where aryl is as defined above. Aryloxy groups cart contain 5 to 20 carbon atoms, and can contain, for example, 5 to 14 carbon atoms. Examples of aryloxy groups incluide, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Examples of aralkyl groups contain 6 to 24 carbon atoms, and particularly preferred aralkyl groups contain 6 to 16 carbon atoms.

The term “cyclic” refers to alicyclic or aromatic, substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen, sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 10 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substitute groups, and the term “heteroatom-containing hydrocarbyl” refers to hydrocarbyl which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, in least one hydrogen atom bound to a carbon (or other) atom is replaced with one or more non-hydrogen substituents. If a particular group permits, it may be further substituted with one or more additional functional groups or with one or more hydrocarbyl moieties. Analogously, the above-mentioned hydrocarbyl moieties may he further substituted with one or more functional groups or additional hydrocarbyl moieties.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

The present invention relates to compounds and methods for promoting differentiation of oligodendrocyte precursors. Compounds in accordance with the invention can be used in the treatment of neurodegenerative disorders, such as multiple sclerosis, to induce and or promote differentiation of oligodendrocyte precursor cells. The term “oligodendrocyte precursor cells” as used herein refers immature oligodendrocyte cells. Oligodendrocyte precursor cells can be identified by the expression of a number of surface antigens. For example, the surface antigens known as platelet-derived growth factor-alpha receptor subunit (PDGFRα), NG2 chondroitin sulfate proteoglycan, and ganglioside GD3, are commonly used to identify oligodendrocyte precursor cells.

Immature oligodendrocyte precursors are generated in ventral areas of the developing brain from a common glial progenitor. The immature cells actively migrate and proliferate populating the CNS to finally differentiate to premyelinating oligodendrocytes (O4+). Oligodendrocyte precursor differentiation and maturation is characterized by an extension of multiple processes, increase in cell body size and formation of myelin.

In one aspect of the present invention, the compounds in accordance with toe present invention are identified using a high-throughput small molecule screen that is biased to identify compounds that have both a high potency and low toxicity in mammal subjects and are able to promote oligodendrocyte precursor differentiation. The term “small molecule” as used herein refers to biologically active organic compounds of low molecular weight (e.g. <500 kDa) which may cross biological membranes and modulate intracellular processes.

Briefly, the high-throughput small molecule screen included a primary screening where small drug-like organic compounds (250-550 kDa) are added to cells seeded on a 96-well plate an incubated. The cells are then visually screened for oligodendrocyte precursor morphology changes, in a secondary screening, differentiation induced by selected compounds was further validated by fluorescence microscopy. Increased fluorescence in treated oligodendrocyte cells generated from a Plp-EGFP transgenic mouse was indicative of cell maturation, Further oligodendrocyte precursor maturation in response to selected compounds was assessed by induction of myelin protein expression as determined by immunocytochemistry and western blot. (see Example 1 below)

Examples of compounds identified by the high-throughput small molecule screen that can be used to promote oligodendrocyte precursor differentiation have the following general formulas:

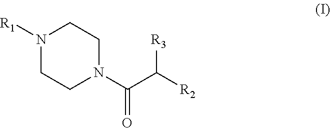

wherein R₁, R₂, and R₃ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, carboxy, a carboxylato, a carbamoyl, an amino, a substituted amino, an alkylamido, an arylamido, an imino, an alkylimino, an arylimino, a nitro, a nitroso, salts thereof, and combinations thereof.

Other examples of compounds identified by the high-throughput small molecule screen that can he used to promote oligodendrocyte precursor differentiation have the following general structure:

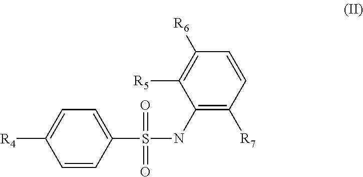

wherein R₄, R₅, R₆, and R₇ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, salts thereof, and combinations thereof.

Examples of compounds having formula I that can be used to promote differentiation of oligodendrocyte precursors include:

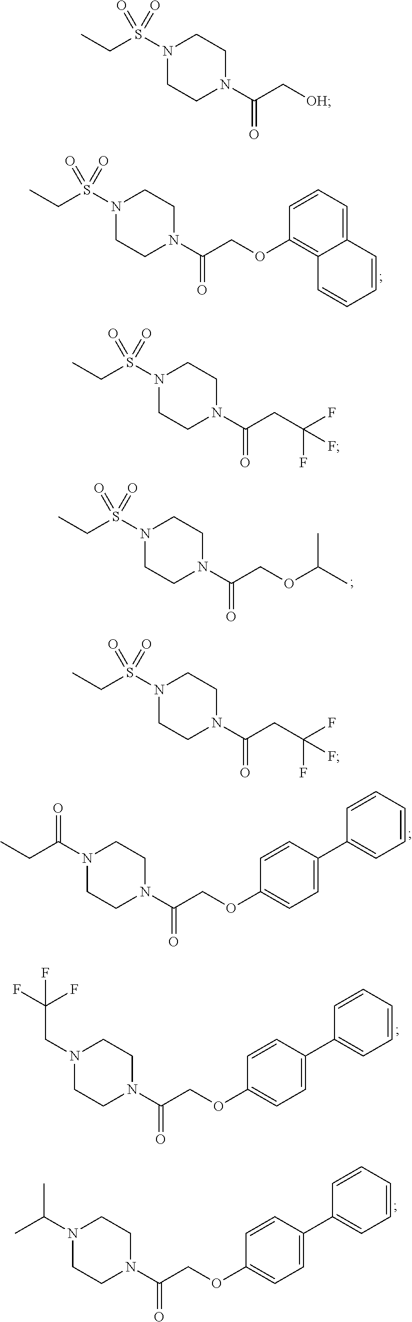

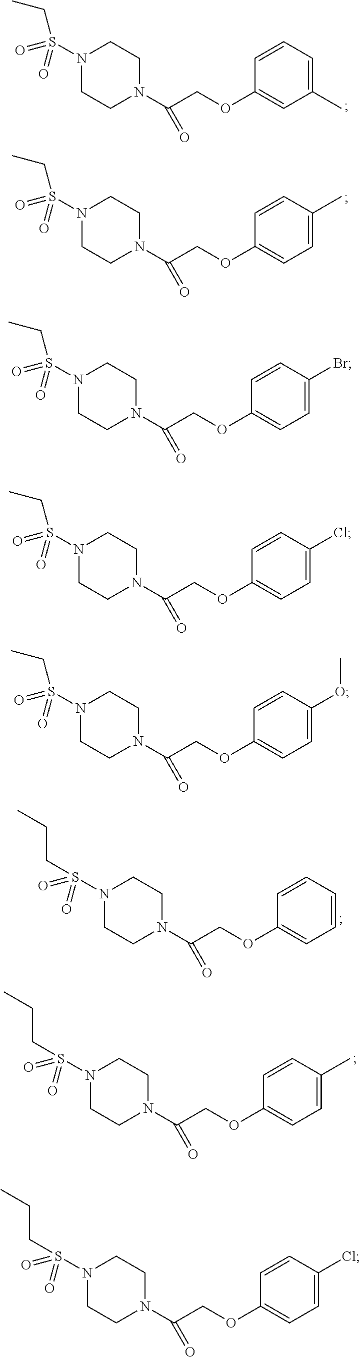

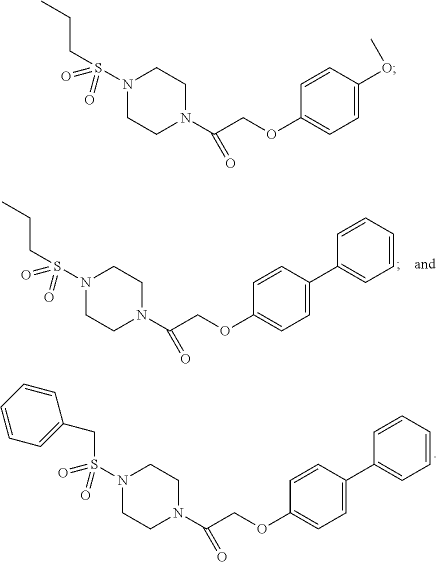

Examples of compounds having formula II that can be used to promote oligodendrocyte precursor differentiation include:

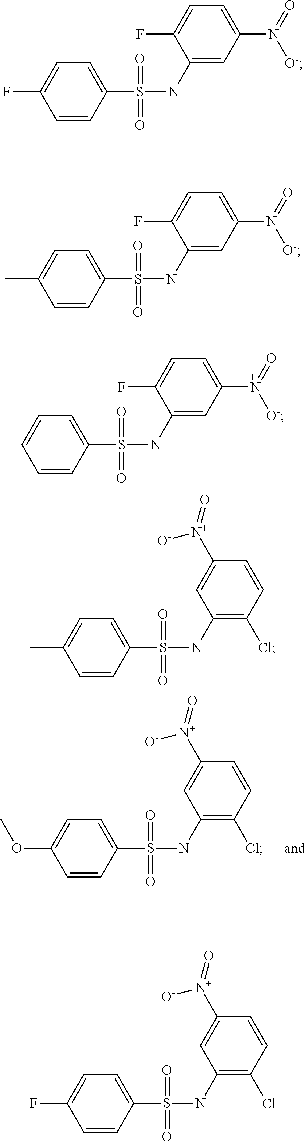

In one specific example, oligodendrocyte precursor differentiation can be provided by administrating to oligodendrocyte precursors an effective amount of a compound having the following structure:

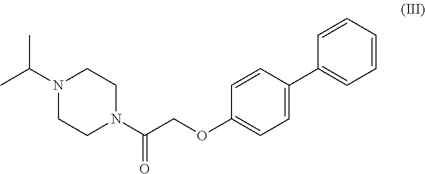

Advantageously, compound III was found to have high solubility, high hydrophobicity, and produce dramatic up-regulation of the myelin protein PLP/DM20 expression compared to other compounds and controls. Compound III was generated according to the synthesis scheme shown in Example 2 below.

When referring to a compound of the invention, applicants intend the term “compound” to encompass not orb the specified molecular entities but also their pharmaceutically acceptable, pharmacologically active analogs, including, but not limited to, salts, esters, amides, prodrugs, conjugates, active metabolites and other such derivatives, analogs, and related compounds.

The oligodendrocyte precursor cell differentiation promoting compounds of the present invention can be provided and administered in the form of pharmaceutical compositions for the in vivo promotion of oligodendrocyte precursor differentiation. The pharmaceutical compositions can be administered to any subject that can experience the beneficial effects of the oligodendrocyte precursor differentiation compounds of the present invention. Foremost among such animals are humans, although the present invention is not intended to be so limited.

Pharmaceutical compositions for use in the methods of the present invention preferably have a therapeutically effective amount of the compound or salts thereof in a dosage in the range of 0.01 to 1,000 mg/kg of body weight of the subject, and more preferably in the range of from about 10 to 100 mg/kg of body weight of the patient.

The overall dosage will be a therapeutically effective amount depending on several factors including the overall health of a subject, the subject's disease state, severity of the condition, the observation of improvements and the formulation and route of administration of the selected agent(s). Determination of a therapeutically effective amount is within the capability of those skilled in the art. The exact formulation, route of administration and dosage can be chosen by the individual physician in view of the subject's condition.

The present invention provides a method of treating diseases in a subject by promoting the differentiation of oligodendrocyte precursors in a subject. The method includes administering to the subject in need thereof a therapeutically effective amount of a pharmaceutical compound in accordance with the present invention. As described above, one or more of the compounds can be administered in association with one or more non-toxic, pharmaceutically acceptable carriers and/or diluents and/or adjuvants and if desired other active ingredients.

The “therapeutically effective amount” of compounds and salts thereof used in the methods of the present invention varies depending upon the manner of administration, the age and body weight of the subject, and the condition of the subject to be treated, and ultimately will be decided by those skilled in the art. The term “therapeutically effective amount” refers to an amount (dose) effective in treating a subject, having, for example, a neurodegenerative disease (e.g. multiple sclerosis).

“Treating” or “treatment” as used herein, refers to the reduction in severity and/or frequency of symptoms, elimination of symptoms and/or underlying cause, prevention of the occurrence of symptoms and/or their underlying cause, and improvement or remediation of disease. Such treatment need not necessarily completely ameliorate the disease. For example, treatment of a subject with a neurodegenerative disease by administration of oligodendrocyte precursor differentiation compounds of the present invention can encompass inhibiting or causing regression of the disease. Further, such treatment can be used in conjunction with other traditional treatments for neurodegenerative diseases known to those of skill in the art.

The pharmaceutical compositions of the present invention can be administered to a subject by any means that achieve their intended purpose. For example, administration can be by parenteral, subcutaneous, intravenous, intraarticular, intrathecal, intramuscular, intraperitoneal, or intradermal injections, or bytransdermal, buccal, oromucosal, ocular routes or via inhalation. Alternatively, or concurrently, administration can be by the oral route.

Formulation of the pharmaceutical compounds for use in the modes of administration noted above (and others) are known in the art and are described, for example, in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y., U.S.A., 2005; and Mathiowitz et al., eds., Bioadhesive Drug Delivery Systems, Drugs and the Pharmaceutical Sciences Series, Marcel Dekker, Inc., N.Y., U.S.A., 1999. Compounds of the invention can be formulated into pharmceutical compositions containing pharmaceutically acceptable non-toxic excipients and carriers. The excipients are all components present in the pharmaceutical formulation other than the active ingredient or ingredients. Suitable excipients and carriers useful in the present invention are composed of materials that are considered safe and effective and may be administered to an individual without causing undesirable biological side effects, or unwanted interactions with other medications. Suitable excipients and carriers are those, which are composed of materials that will not affect the bioavailability and performance of the agent. As generally used herein “excipient” includes, but is not limited to surfactants, emulsifiers, emulsion stabilizers, emollients, buffers, solvents, dyes, flavors, binders, fillers, lubricants, and preservatives. Suitable excipients include those generally known in the art such as the “Handbook of Pharmaceutical Excipients”, 4th Ed., Pharmaceutical Press, 2003.

The compounds in accordance with the present invention can be administered to a subject to treat neurodegenerative conditions. A neurodegenerative disease, as contemplated for treatment by methods of the present invention, can arise from but is not limited to stroke, heat stress, head and spinal cord trauma (blunt or infectious pathology), and bleeding that occurs in the brain. Examples of neurodegenerative disorders contemplated include Alexander disease, Alper's disease, Amyotrophic lateral sclerosis, Ataxia telangiectasia, Spielmeyer-Vogt-Sjogren-Balten disease, Bovine spongiform encephalopathy. Canavan disease, Cockayne syndrome, Corticobasal degeneration, Creutzfeldt-Jakob disease, Huntington's Disease, HIV-associated dementia, Kennedy's disease, Krabbe disease, Lewy body dementia, Spinocerebellar ataxias, Multiple Sclerosis, Multiple system atrophy, Neuroborreliosis, Parkinson's disease, Pelizaeus-Merzbacher disease, Pick's disease, Primary lateral sclerosis, Prion diseases, Refsum's disease, Sandhoff disease, Schilder's disease, Spinal muscular atrophy, Steele-Richardson-Olszewski disease, and tabes dorsalis.

The neurodegenerative disease contemplated for treatment by some aspects of the present invention can include a myelin related disorder. Myelin disorders can include any disease, condition (e.g., those occurring from traumatic spinal cord injury and cerebral infarction), or disorder related to demylination, remylination, or dysmyelination in a subject. A myelin related disorder as used herein can arise from a myelination related disorder or demyelination resulting from a variety of neurotoxic insults. “Demyelination” as used herein, refers to the act of demyelinating, or the loss of the myelin sheath insulating the nerves, and is the hallmark of some neurodegenerative autoimmune diseases, including multiple sclerosis, transverse myelitis, chronic inflammatory demyelinating polyneuropathy, and Guillain-Barre Syndrome. Leukodystrophies are caused by inhumed enzyme deficiencies, which cause abnormal formation, destruction, and/or abnormal turnover of myelin sheaths within the CNS white matter. Both acquired and inherited myelin disorders share a poor prognosis leading to major disability. Thus, some embodiments of the present invention can include methods for the treatment of neurodegenerative autoimmune diseases in a subject. The term “remyelination”, as used herein, refers to the re-generation of the nerve's myelin sheath by replacing myelin producing cells or restoring their function.

One particular aspect of the present invention contemplates the treatment of multiple sclerosis in a subject. The method includes administering to the subject a therapeutically effective amount of one or more oligodendrocyte differentiation promoting compound(s) described above.

Multiple sclerosis (MS) is the most common demyelinating disease. In multiple sclerosis, the body's failure to repair myelin is thought to lead to nerve damage, causing multiple sclerosis associated symptoms and increasing disability. It is contemplated that methods of the present invention can promote oligodendrocyte precursor cell differentiation in a subject, therefore leading to endogenous remyelination.

Another strategy for treating a subject suffering from a neurodegenerative disease or disorder is to administer a therapeutically effective amount of a compound described herein along with a therapeutically effective amount of additional oligodendrocyte differentiation inducing agent(s) and/or anti-neurodegenerative disease agent. Examples of anti-neurodegenerative disease agents include L-dopa, cholinesterase inhibitors, anticholinergics, dopamine agonists, steroids, and immunomodulators including interferons, monoclonal antibodies, and glatiramer acetate.

Therefore, in a further aspect of the invention, the oligodendrocyte precursor differentiation inducing agents can be administered as part of a combination therapy with adjunctive therapies for treating neurodegenerative and myelin related disorders.

The phrase “combination therapy” embraces the administration of the oligodendrocyte precursor differentiation inducing agents and a therapeutic agent as part of a specific treatment regimen intended to provide a beneficial effect from the co-action of these therapeutic agents. When administered as a combination, the oligodendrocyte precursor differentiation inducing agents and a therapeutic agent can be formulated as separate compositions. Administration of these therapeutic agents in combination typically is carried out over a defined time period (usually minutes, hours, days or weeks depending upon the combination selected).

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination ma be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to a second and different therapeutic agent) and non-drug therapies (e.g., surgery).

In another aspect of the invention, the therapeutic agents administered in a combination therapy with the oligodendrocyte differentiation inducing agents can include at least one anti-neurodegenerative agent selected from the group consisting of an immunotherapeutic agent.

An immunotherapeutic agent for use in the methods of the present invention can include therapies which target the immune component of the disease and/or the acute inflammatory response evidenced during an acute attack in remitting-relapsing multiple sclerosis. Examples include, but are not limited to immunomodulators such as interferon beta-1a and beta-1b (Avonex and Betaseron respectively), natalizumab (Copaxone) natalizumab (Tysabri), glatiramer acetate (Copaxone) or mitoxantrone.

It should be understood that the methods described herein may be carried out in a number of ways and with various modifications and permutations thereof that are well known in the art. It may also be appreciated that any theories set forth as to modes of action should not be construed as limiting this invention in any manner, but are presented such that the methods of the invention can be more fully understood.

The following examples further illustrate aspects of the present invention. However, they are in no way a limitation of the teachings or disclosure of the present invention as set forth herein. Other suitable modifications and adaptations of the variety of conditions and parameters normally encountered and obvious to those skilled in the art are within the spirit and scope of the invention.

The contents of all references, patent applications, patents, and published patent applications cited throughout this application are hereby incorporated by reference in their entirety.

### EXAMPLE 1

In this Example, we identified a series of small molecules able to induce differentiation in cultured oligodendrocyte progenitors and myelin formation in demyelinated tissue explants. The most effective compounds have been tested for their effect on oligodendrocyte maturation in zebrafish and, more importantly, we have found a remarkable efficacy in enhancing oligodendrocyte remyelination in a mouse model of demyelination.

### Methods and Materials

### Animals

For in vivo experiments in mice, the cuprizone-mediated demyelination model was implemented as described elsewhere, (Sun et al., 2006; Wu et al., 2008). In brief, 9 week-old C57B1/J6 mice were fed with cuprizone supplemented chow (0.3%, Harlan) during 4-6 weeks. At the end of this period, while the mice were still on cuprizone diet, the animals were injected I.P. with the indicated concentrations of small molecules dissolved in DMSO. 72 hrs later, a second dose was administered and at that point the food was changed to regular chow. 48 hr after the second dose, the animals were sacrificed perfused with 4% paraformaldehyde (PFH) and the brain tissue processed or immunohistochemistry.

### Cell Cultures

Oligodendrocyte progenitors were generated from embryonic neural progenitors growing in cell aggregates known as neurospheres (Nphs). Nph were prepared from embryos at 14.5 days of gestation obtained from timed pregnant females of either wild type mice or the proteolipid protein-enhanced-green fluorescence protein (Plp-EGFP) transgenic mouse. In brief, after separation of meninges and cerebellum, cerebrum tissue was mechanically triturated with a 1 ml Gilson pipette until total tissue disaggregation was achieved, filtered through a 70 μm cell strainer (Fisher Scientific, Pittsburg, Pa.) and plated in 25 cm² plastic culture flasks (2 brains/flask). Nph proliferation media (NPM) was DMEM/F12, B27 neuronal supplement (Gibco, Baltimore, Md.) and 10 ng/ml EGF (Sigma, St. Louis, Mo.). After 48-72 hr, floating Nphs were passaged at a 1:3 ratio in the same medium every 3-4 days. To generate oligodendrocyte progenitors, single cells suspensions were obtained by chemical disgregation with the mouse NeuroCult dissociation kit (StemCell Technologies, Alberta, Canada). The single cell suspension was filtered through a 70 μm cell strainer and plated on 96-well poly-D-Lysine (PDL)-coated plates at a cell density of 1.5×10⁴ cells/cm². Cells were maintained in NPM supplemented with 10 ng/ml PDGF/bFGF instead of EGF. Bipolar, oligodendrocyte progenitor cells were observed 24 hours after plating. At the Moment of treatment to induce oligodendrocyte progenitor differentiation, the media was changed to OPC differentiation media containing NPM with a 1:10 dilution of PDGF/FGF.

### Cerebellar Explants

Myelination-demyelination-remyelination studies in cerebella organotypic cultures were carried out. In brief, after careful dissection to keep the tissue intact, cerebella of postnatal day 4-7 wild type or Plp-EGFP mouse pups was sectioned at 400-μm thickness using a Leica VT1000S vibratome. Cerebella sections were then plated on insert plates and a few droplets of high-glucose media were added to cover the tissue during the first 6 hours to allow attachment of the slices to the insert plate surface. Media used was 50% DMEM/F12, 25% Hank's buffered salt solution (both from Gibco), 25% horse serum, 5 mg/ml glucose and a combination of streptomycin/penicillin (Gibco). When the slices were firmly attached, fresh media was added to cover the tissue and the cultures were maintained in this conditions during 4-10 days. To induce demyelination, 0.5 mg/ml lysolecithin (I′-monoacyl-L-3-glycerylphosphorylcholine, Sigma) was added to the culture media for 17 hrs. After this time, the culture media was changed and treatments were performed by adding small molecules or DMSO controls to the plates for 4-10 additional days. Demyelination-remyelination was assessed by green fluorescence (Plp-EGFP) or immunostaining for myelin-axonal proteins and visualization was performed by confocal microscopy.

### Zebra Fish Oligodendrocyte Development

Transgenic-Proteolipid protein-enhanced green fluorescence protein (tg[plp:EGFP]) zebrafish was raised and maintained (Yoshida and Macklin, J. Neurosci Res, 81:1, 2005). Embryos were collected and staged by hour post fertilization (hpf). Compounds were added at 24 hpf stage at a concentration of 10 nM, 100 nM and 1 μM. After 48 hrs (72 hpf stage), zebrafish embryos were anesthetized in 0.02% tricaine, fixed in 4% paraformaldehyde, and washed in phosphate buffered saline (PBS). After treating with 10% Triton X-100 for 30 minutes, embryos were washed in PBS and blocked with 3% goat serum. Embryos were then incubated at 4° C. overnight with FITC-conjugated goat anti-GFP antibody (Abcam, Cambridge, Mass.) at a concentration of 1:1000. After several washes with PBS, the embryos were embedded in 100% glycerol and visualized on a Leica SP5 confocal microscope.

### Small Molecule High-Throughput Screening Methodology

### Primary Screening

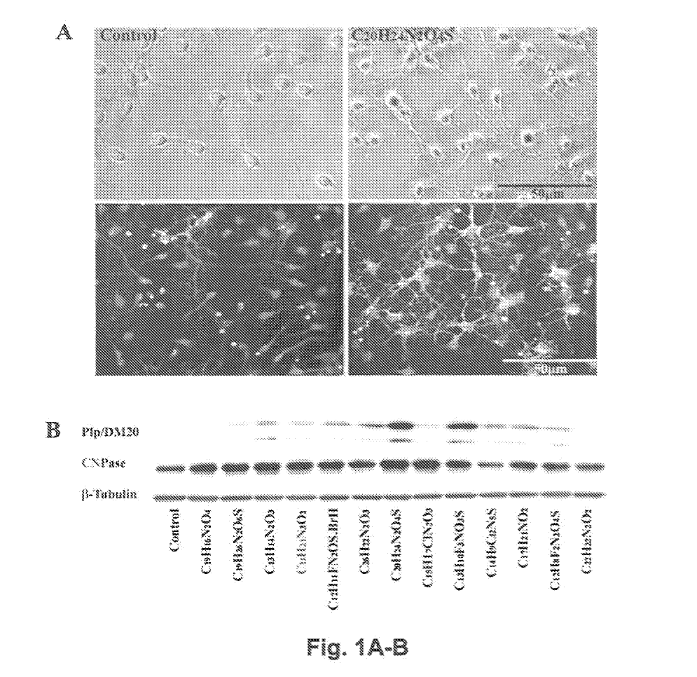

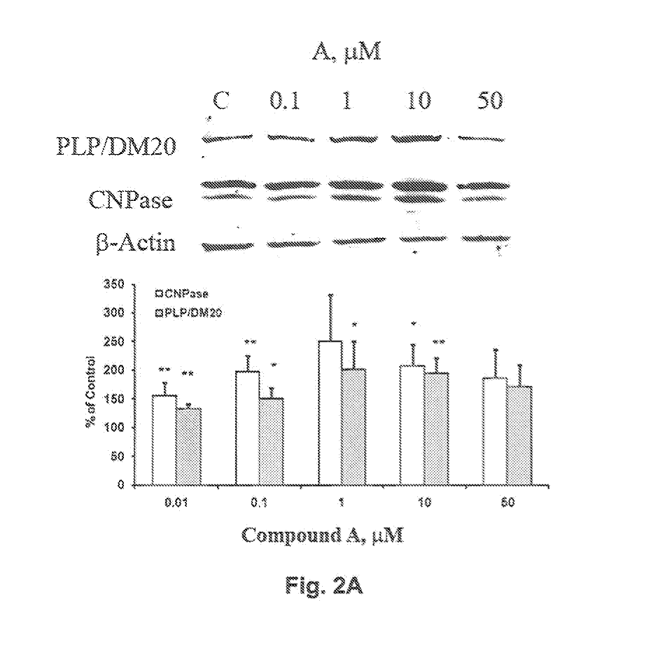

The small molecule library consists of handcrafted drug-like organic molecules with molecular weight in a range of 250-550, dissolved in DMSO at concentration of 5 mg/ml or 10 M. Their structure and >95% purity have been validated by NMR. The library is formatted in 96-well plates with 80 compounds per plate. 16 wells on the lateral edges contained only DMSO and were used for controls (Chembridge, Boston, Mass.). Cells were seeded on 96-well plates with OFC differentiation media (200 μl/well). Cells were treated with 0.2 μl of small molecule solutions to a final concentration of 10 μM, for 48 h. After this incubation time visual screening for oligodendrocyte progenitor morphology changes was performed under a phase contrast microscope. Oligodendrocyte progenitor differentiation was scored using as 4 scale values as follows: 1: bipolar, undifferentiated; 2: bipolar to tripolar, initiated differentiation; 3: multibranched, differentiated cells; 4: multibranched, differentiated cells showing membranous structures (FIG. 1).

### Secondary Screening

Differentiation induced by selected compounds was further validated by fluorescence microscopy in cells generated from our Plp-EGFP transgenic mouse. Increased fluorescence in treated oligodendrocyte progenitor was indicative of cell maturation and was again scored following the same scale. 13 positive compounds were identified after secondary screening (FIG. 1). These were analyzed for cell toxicity assays by MTT were yellow 3-(4,5-Dimethylthiazol-2-yl)-2,5-diphenyltetrazolium bromide, a tetrazole (MTT) is reduced to purple formazan in the mitochondria of living cells, showing a solution color change measurable by OD620-590 (Data not shown). Further oligodendrocyte progenitor maturation in response to selected compounds was assessed by induction of myelin protein expression as determined by immunocytochemistry and western blot.

### Immunostaining and Black and Gold Staining for Total Myelin

Immunodetection of oligodendrocyte and myelin markers was performed. In brief, Oligodendrocyte cells plated on PDL-coated dishes were initially fixed in 2% paraformaldehyde (PFA) for 10 min at room temperature (RT) and then 4% PFA for 15 min, washed three times with PBS and permeabilized with 0.1% Triton X100 for 10 min at RT. After blocking with 3% BSA in PBS for 60 min a RT, the cells were incubated with primary antibodies at the indicated concentrations overnight (ON) at 4° C. The cells were washed three times with cold PBS incubated with secondary antibodies for 45 min at RT, and washed again before mounting. Immunostaining of tissue sections prepared with a sliding microtome (30 μm) was performed. In brief, tris buffered saline (TBS) containing 100 nM Sodium fluoride (TBSS) was used the all dilutions and washes. For antigen retrieval, sections were washed 3 times in TBSS, incubated in 5% methanol and 3% H₂O₂ for 10 min, washed 2 times, and then incubated in 10% Triton X-100 for 20 min. Blocking of unspecific bonds was performed by incubating the tissue sections with 10% NGS for 1 hr. Primary antibodies were diluted in 3% BSA containing 0.02% Triton X-100 and sections were incubated overnight in this solution. The next day the sections were washed 3 times in TBSS, followed by 1 hr incubation in secondary antibodies (1:1000) at 4° C. After several washes, the sections were mounted using Vectashield (Vector Laboratories, Burlingame, Calif.). Visualization of immunostained cells and tissue was performed on a Leica DMR (Wetzlar, Germany) or a Leica SP5 confocal microscope.

Brain tissue staining with Black and Gold to detect total myelin was performed in 30 μm microtome floating sections after three washes in a saline solution (0.9% NaCl). Subsequently, the tissue sections were submerged in a 0.1% Black and Gold solution in saline preheated to 60° C. for 5 min. The tissue was incubated in that solution for 8-10 min. or until achievement of purple color was observed. After washing several times with saline, the staining was fixed with a solution of 2% sodium thiosulfate for 3 min., washed with saline three times and finally mounted and visualized.

Primary antibodies used in this study included rat anti-proteolipid (PLP)/DM20 (1:1000, clone AA3), anti-2′,3′-Cyclic Nucleotide 3′-Phosphodiesterase and anti-Myelin Oligodendrocyte Glycoprotein (MOG) (Abeam, Cambridge, Mass.), anti-Myelin Basic Protein (Chemicon), mouse anti-β-III-tubulin (1:300, Chemicon, Temecula, Calif.), rabbit anti-PDGFR-α (1:300, Santa Cruz, Calif.), anti-rat, anti-rabbit or anti-mouse AlexaFluor secondary antibodies (Gibco) were used at 1:700. Anti-rat, anti-rabbit or anti-mouse IRdye infrared conjugated secondary antibodies for densitometry analysis using the Odyssey system were used at 1; 20.000 dilutions.

### Western Blot

For myelin protein analyses, cells were grown on 100 mm culture plates and after exposure to small molecules or controls were washed twice with ice-cold PBS. RIPA lysis buffer was added to the plates and the cells were scraped, collected and disrupted with a homogenizer on ice. Cell homogenates were then centrifuged at 6,000×g, 10 min, 4° C. and supernatants separated in a fresh tube. After adding Laemli loading buffer, proteins were denaturized at 70° C. for 10 mm and loaded in gradient gels (4-20, Criterion, BioRad, Calif.). Electroforetically separated proteins were electro-transferred to nitrocellulose membranes in a semi-dry apparatus and unspecific bonds were blocked with IRdye blocking solution for at least 1 hr at RT. After washing twice with TBS-T, primary antibodies were added and incubated over night at 4° C. Membranes were incubated with secondary, IRdye-infrared conjugated antibodies for 1 hr at RT and visualized on the Odyssey scanner.

### Detection of Small Molecules in Blood Plasma and Brain Tissue by Mass Spectrometry

Samples of blood plasma and total brain homogenates from four mice injected with compound A and A-7 were analyzed for the content of these compounds by mass spectrometry. The signal was optimized for each compound for positive or negative ionization by ESI (Electrospray ionization). A standard curve what made with pure compound solutions in DMSO up to 10 ng/ml. A MS2 scan was used to identify the progenitor ion and a product ion analysis was used to identify the best fragment for analysis and to optimize the collision energy. An ionization ranking was assigned indicating the compound's ease of ionization. Samples were precipitated in 3 volumes of methanol with internal standard, centrifuged, and filtered. Filtered samples were analyzed by LC/MS/MS using either an Agilent 6410 mass spectrometer coupled with an Agilent 1200 HPLC and a CTC PAL chilled autosampler, all controlled by MassHunter software (Agilent), or an AB12000 mass spectrometer coupled with an Agilent 1100 HPLC and a CTC PAL chilled autosampler, all controlled by Analyst software (ABI). After separation on a C18 reverse phase HPLC column (Agilent, Waters, or equivalent) using an acetonitrile-water gradient system, peaks were analyzed by mass spectrometry (MS) using ESI ionization in MRM (Multiple Reaction Monitoring) mode.

### Results

### Small Molecule Screening

The main goal of this work was to identify new compounds inducing oligodendrocyte progenitor differentiation from a small molecule library. To this end and taking advantage of the marked cell morphology changes oligodendrocyte lineage cells undergo from bipolar, fusiform progenitors to multi-branched premyelianting oligodendrocytes, we established a cell morphology-based screening. Thus, we set up a primary screening based on cell visualization under a phase-contrast microscope which as later complemented with the analysis of green fluorescence m oligodendrocyte progenitors form our Plp-EGFP mouse. The available small molecule library contained 34,000 compounds from the Chembridge library, which could be expanded to +250,000 small molecules. To produce enough cells to cover such a big number of small molecules, we established a new methodology to generate oligodendrocyte progenitor cells from neural progenitors with high purity and experimental reproducibility (see Methods and Pedraza, et al. 2008).

Differentiation was scored as cells changing from a bipolar, fusiform shape to a multiprocess, membranous morphology using a 1-4 scale: 1 and 2: bipolar to trypolar cells; 3: cells with more than three processes: and 4; cells showing numerous processes and membranous structures (FIG. 1). Thus, differentiated cells were easily identifiable since cells in the negative control (OPC-differentiation media, see Methods) showed the typical bipolar, elongated oligodendrocyte progenitor morphology (1-2 scores. FIG. 1) and cells undergoing differentiation showed multiple processes and membranous structures. As positive control for cell differentiation in our primary screening we used ciliary neurotrophic factor (CNTF, 10 ng/ml, 48 hrs). This neurotrophin induced marked oligodendrocyte progenitor morphological changes indicative of cell maturation (not shown). Additionally, green fluorescence in cells generated from our Plp-EGFP transgenic mouse (Mallon et al., 2002, data not shown) and immunostaining for the differentiated oligodendrocyte proteins, proteolipid protein (PLP) and myelin basic protein (MBP), were used to confirm increased PLP expression as a measurement of oligodendrocyte progenitor differentiation (FIG. 1).

From the primary screening of 13,920 small molecules, we found that 87.2% had no apparent effect, 12.8% induced cell death and 0.01% induced clear morphology changes and increased green fluorescence in Plp-EGFP oligodendrocyte progenitors. From subsequent secondary screenings of positive hits, we identified 13 compounds that were analyzed further (Table 1, FIG. 1, B).

TABLE 1

| Formula | MW | IUPAC Name |
|---|---|---|
| C19H16N2O4 | 336.3 | 4-(1,3-benzodioxol-5-ylmethylene)-1-(3,4-dimethylphenyl)-3,5-pyrazolidinedion |
| C19H26N2O6S | 410.4 | 2-(ethylthio)ethyl 6-methyl-2-oxo-4-(3,4,5-trimethoxyphenyl)-1,2,3,4-pyrimidine |
| C13H14N2O3 | 246.2 | (4-oxo-3-propyl-3,4-dihydro-1-phthalazinyl)acetic acid |
| C11H21N3O2 | 227.3 | N-isopropyl-2-[2-(1-methylpentylidene)hydrazino]-2-oxoacetamide |
| C12H11FN2OS.BrH | 331.2 | 3-(3-fluoro-4-methoxyphenyl)-5,6-dihydroimidazo[2,1-b][1,3]thiazole hydrobromide |
| C26H22N2O3 | 410.4 | ethyl 2-({[2-(2-methylphenyl)-4-quinolinyl]carbonyl}amino)benzoate |
| C20H24N2O4S | 388.4 | 1-[(4-biphenylyloxy)acetyl]-4-(ethylsulfonyl)piperazine → A |
| C15H17ClN2O3 | 308.7 | N-[2-(4-chlorophenyl)-1-(4-morpholinylcarbonyl)vinyl]acetamide |
| C13H10F3NO3S | 317.2 | methyl 4-oxo-2-{[3-(trifluoromethyl)phenyl]amino}-4,5-dihydro-3-thiophenecarboxyl |
| C14H9Cl2N5S | 350.2 | 5-(2,4-dichlorophenyl)-4-[(3-pyridinylmethylene)amino]-4H-1,2,4-triazole-3-thiol |
| C17H21NO2 | 271.3 | N-bicyclo[2.2.1]hept-2-yl-3-(4-methoxyphenyl)acrylamide |
| C12H8F2N2O4S | 314.2 | 4-fluoro-N-(2-fluoro-5-nitrophenyl)benzenesulfonamide → B |
| C22H22N2O2 | 346.4 | 4-(4-morpholinylmentyl)-N-1-naphthylbenzamide |

Primary and secondary screenings were performed in oligodendrocyte progenitors generated from embryonic neurospheres. As an additional confirmation of the oligodendrocyte differentiation effect induced by the selected compounds observed on cells from embryonic origin, we treated cell cultures of oligodendrocyte progenitors generated from post-natal rat and mouse mixed glial cultures. As expected, the differentiation effect of the selected small molecules was also observed in these systems (data not shown).

Two lead compounds were identified form the small molecule screening and will be referred to as A (C20 H24N2 O4 S) and B (C12 H8 F2 N2 O4 S). Furthermore, as a result of medicinal chemistry, database search for structural analogues and experimental functional analysis, one more compound derivative from lead A was also studied in deep. This compound will be referred to as A-7 (C21 H26 N2 O2).

### Dose and Time-Dependency Effects of Selected Small Molecules

The primary screening to select positive compounds was performed at a standard concentration of 10 μM with cell morphology analysis 48 hrs after treatment. Dose response curves at 48 hrs treatment time were performed to determine the minimum concentration compounds A, A-7 and B needed to induce cell maturation. We assessed oligodendrocyte differentiation by cell morphology changes, green fluorescence in cells generated from or Plp-EGFP transgenic mouse, immunostaining for myelin proteins and western blot for expression of the oligodendrocyte markers PLP/DM20 and 2′,3′, cyclic nucleotide 3′ phosphohydrolase (CNPase). EC50 values were also calculated based on results of western blot for myelin proteins and subsequent band densitometry analysis. Additionally, we followed cell differentiation induced over long time exposure to compounds A and B (7 days, 0.1-10 μM).

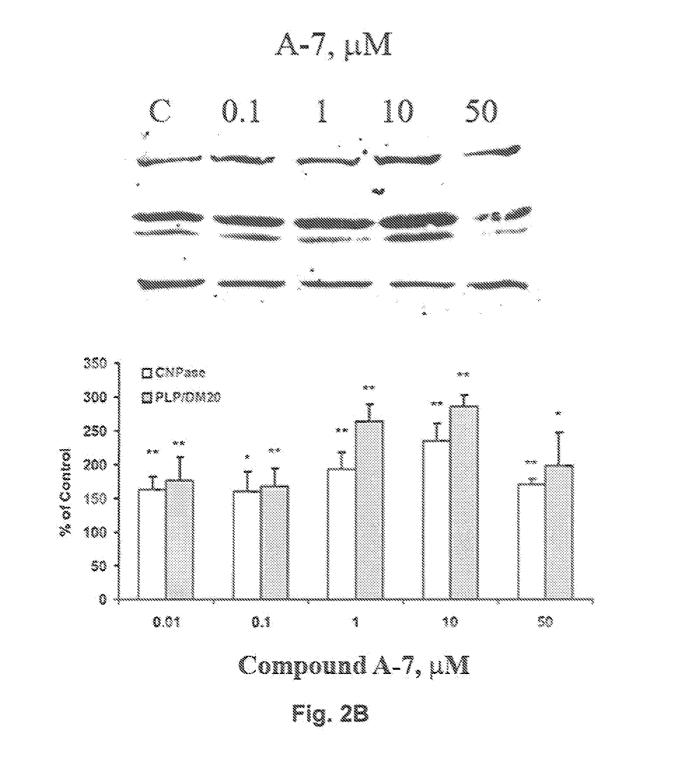

Treatment of oligodendrocyte progenitor cells with compound A increased expression of the major myelin proteins already detectable a low concentrations (0.1 μM) being greatly increased at 1-10 μM (FIG. 2, A, top row). However, highly statistically significant differences were determined from western blots, after band densitometry analysis, for cells treated with concentrations ranging from 0.01 to 10 μM (FIG. A, bottom row). Compound B, on the other hand, was more effective in inducing myelin protein expression at low concentrations (0.01-1 μM). High doses of compound B (10-50 μM) did not induce higher oligodendrocyte cell differentiation than that observed for control cells (FIG. 2, A). Correspondingly, we observe induction of cell death at high concentrations of compound B indicating a toxic effect of this compound at concentrations superior to 10 μM (Data not shown). Statistical analysis from 4 independent experiments confirmed the significant effect of compound B at low concentrations (FIG. 2, A), EC50 values for compounds A and B according to myelin protein expression, as determined by western blot, were 0.023 and 0.007 (μM, 48 hrs for PLP/DM20) and 0.01 and 0.005 (μM, 48 hrs for CNPase) respectively.

Additionally, we analyzed by immunocytochemistry for myelin proteins, the cell responses to long-term exposure to compounds A and B (7 days after treatment). Compound A treated cells showed clear morphology changes with generation of thick, multiple processes and membranous structures. At this time point, about 25% of the total cells were found k be differentiated in response to 0.1-10 μM compound A, versus 9.5% in the control conditions. Compound B, on the other hand, induced clear morphology changes in response to the lowest concentrations (0.1 μM). At 10 μM the cells appeared less differentiated by morphology and the percentage of mature cells dropped to control levels (FIG. 2. B histogram). MTT assays to determine cell viability further confirmed the toxic effect of compound B at concentrations higher than 1 μM (Data not shown).

### Compound Optimization and Structure-Activity Relationship Analyses

Advanced medicinal chemistry to identify biological active compounds sharing chemical structure analogy with the leads A and B was performed. The main objectives of this chemical analysis were to identify the pharmacophore structure and to find compounds with enhanced biological activity based on the lead's chemical scaffold structure. We screened 19 analogue chemicals to compound A and 6 to compound B for their ability to induce PLP/DM20 expression and cell differentiation as determined by western blot and immunohistochemistry respectively. (Tables 2A-C).

TABLE 2, A
Compound A

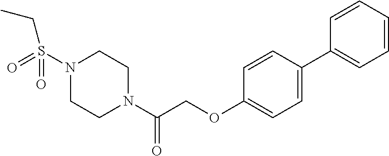

| | |
|---|---|
| 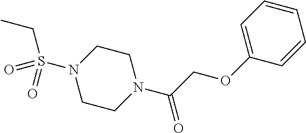 | A-8 |
| 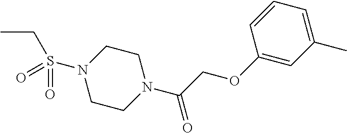 | A-9 |
| 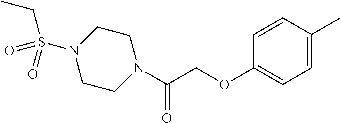 | A-10 |
| 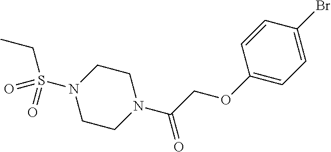 | A-11 |
| 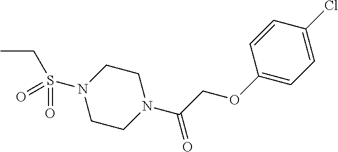 | A-12 |
| 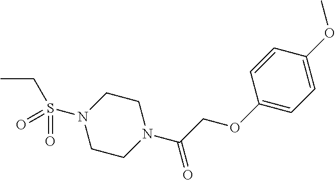 | A-13 |
| 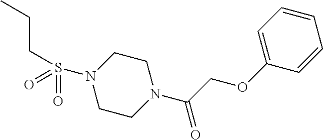 | A-14 |
| 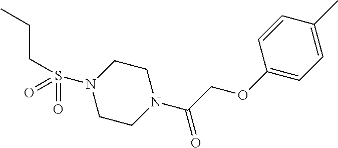 | A-15 |
| 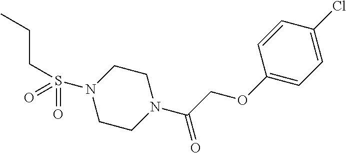 | A-16 |
| 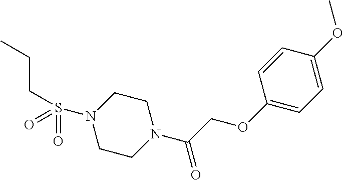 | A-17 |
| 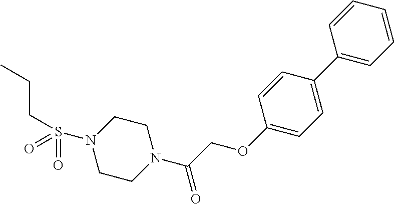 | A-18 |
| 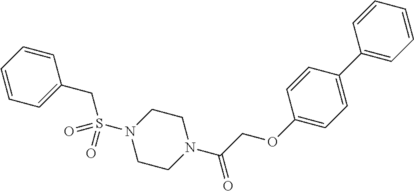 | A-19 |

TABLE 2, B
Compound B

[symbol]

| | |
|---|---|
| 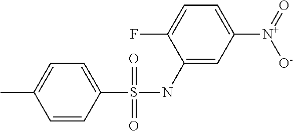 | B-2 |
| 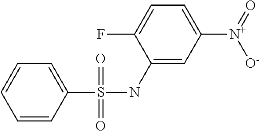 | B-3 |
| 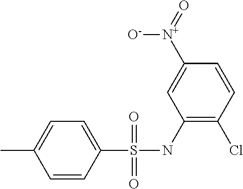 | B-4 |
| 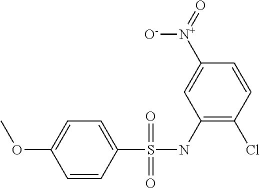 | B-5 |
| 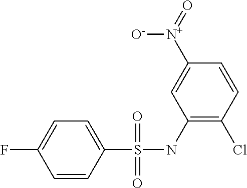 | B-6 |

[symbol]

indicates data missing or illegible when filed

TABLE 2, C
Compound A

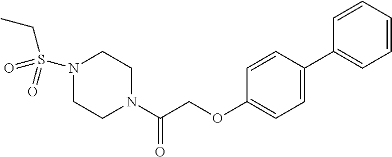

Scaffold A

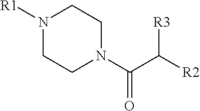

| | |
|---|---|
| 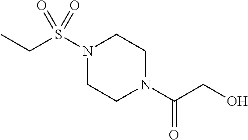 | A-1 |
| 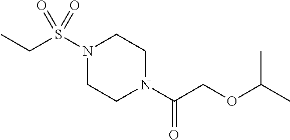 | A-2 |
| 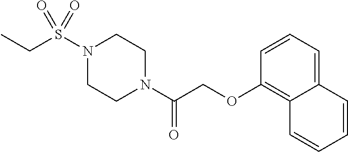 | A-3 |
| 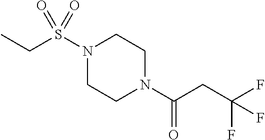 | A-4 |
| 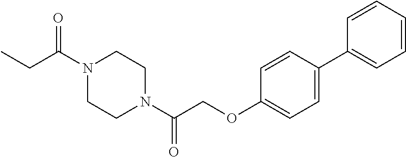 | A-5 |
| 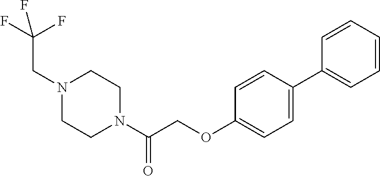 | A-6 |
| 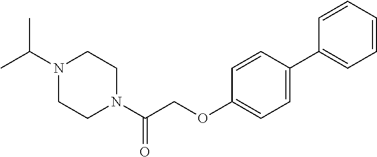 | A-7 |

Compound B analogues tested in this study, did not show relevant improvements when compared with the effect of the lead compound. On the other hand, 9 analogues of compounds A showed unambiguous induction of both cell differentiation and PLP/DM20 expression at similar or improved levels of the lead compound A (Data not shown). Upon further experimentation performing multiple assays, 4 analogues showed enhanced effect in a consistent, reproducible manner. Table 2 summarizes the biological activity of compound A and the analogues showing enhanced biological activity (10 μM, 48 hrs) in terms of expression of PLP/DM20 and induction of cell differentiation determined by immunostaining for PLP/DM20 and MBP. Noticeably, at the concentration of 10 μM, compound A-7 showed the strongest biological activity which accounted for 147% increase in the induction of PLP/DM20 expression levels of the A treated cells and 286.8% of control cells. Correspondingly, a significant increase in the percentage of differentiated cells was observed in response to compound A-7 a compared to A treated cells (159.1%) being even more dramatic an increase (296.1%) when compared to control cells (Tables 3 and 4).

TABLE 3

| | | Bio-activity | |
|---|---|---|---|
| | | 10 μM | |
| | | PLP/DM20 Expression | Differentiated Cells |
| | Formula | (WB, % of Control) | (IC, % of Control) |
| 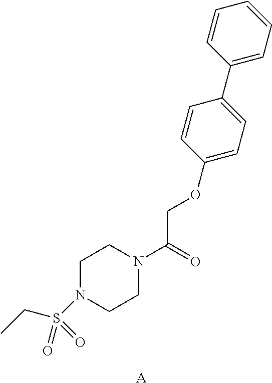 | C₂₀H₂₄N₂O₄S | 195 ± 25.8 n = 6; p <0.01 | 186.1 ± 44.8 n = 4; p <0.05 |
| 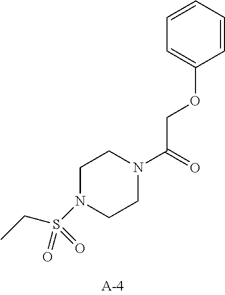 | C₁₄H20N₂O₄S | 148 ± 10.6 n = 2 | 195 ± 10.9 n = 3; p <0.05 |
| 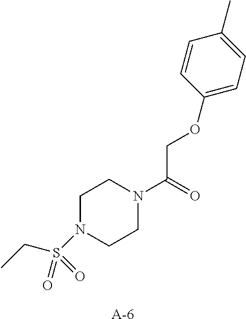 | C₁₅H₂₂N₂O₄S | 160.7 ± 8.2 n = 2 | 207.5 ± 38.2 n = 3; p <0.05 |
| 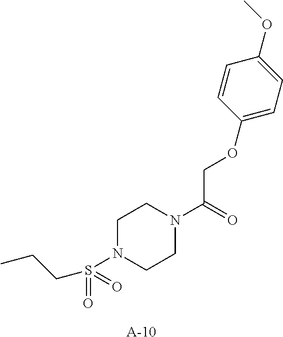 | C₁₆H₂₄N₂O₅S | 143.0 ± 6.9 n = 2 | 224.3 ± 66.3 n = 3; p <0.05 |
| 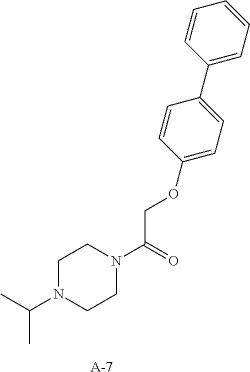 | C₂₁H₂₆N₂O₂ | 286.8 ± 16.5 n = 6; p <0.01 | 296.1 ± 19 n = 4; p <0.05 |

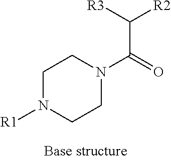

TABLE 4

| | PLP/DM20 Expression | | Differentiated Cells | |
|---|---|---|---|---|
| | (WB, % of Control) | | (IC, % of Control) | |
| | 1 μM | 10 μM | 1 μM | 10 μM |
| A | 201.2 ± 49.4 | 195.1 ± 25.8 | 248.9 ± 47.5 | 289.3 ± 66.6 |
| A-7 | 263.3 ± 25.2 | 286.8 ± 16.5** | 343.2 ± 52.3** | 296 ± 38.06 |

Compound A-7 not only showed enhanced biological activity, but was also more potent since concentrations as low as 0.1 μM induced even stronger effects on oligodendrocyte progenitor cells than those observed in response to compound A at its maximum strength. Table 4 summarizes a comparative analysis of the biological effects of compounds A versus compound A-7 at lower concentrations (0.1 and 1 μM). The expression of PLP/DM20 induced by compound A-7 at 1 μM (263.3%) was higher than that induced by compound A at 10 μM (195.1%)(Table 4, FIG. 2, A) and the number of differentiated cells increased significantly at the same low concentration of compound A-7 greatly augmenting to 343% the control levels at 1 μM. Thus, the amount of compound A-7 necessary to induce the same effect that 10 μM compound A is reduced by 1-2 orders of magnitude. The effect of compound A-7 at 50 μM slightly decreases indicating a potential negative effect on cell viability as the dose increases. However, cell toxicity and viability assays did not show significant detrimental effects of compound A or A-7 at concentrations below 50 μM (data not shown). FIG. 2, A shows a representative western blot for the myelin proteins PLP/DM20 and CNPase in cells treated with increasing concentrations of compound A-7. Quantification by band densitometry of 3-6 independent experiments demonstrated the superior potency of compound A-7 in inducing this effect (FIG. 2, A histogram). EC50 values for A-7 for PLP/DM20 and CNPase expression expression were 0.03 and 0.07 (μM, 48 hrs) respectively.

Compound A-7-induced oligodendrocyte progenitor maturation was also analyzed in comparison to control cells in immunostaining experiments to detect the myelin proteins MBP and PLP. Positive cells for these markers showing processes formation and membranous structures were counted versus total cells and counterstained with the nuclear dye Dapi after 48 hrs (Tables 3 and 4) and 7 days treatment with 0.1, 1 or 10 μM compound A-7 (FIG. 2, B). Once again, compound A-7 showed a overall highly significant induction of oligodendrocyte differentiation, which reached maximum levels at concentrations as low as 0.1 μM (Table 3, FIG. 2, B).

A feasible effect of small molecules on oligodendrocyte progenitor cells is the induction of proliferation. We studied this possibility by treating cultures of oligodendrocyte progenitors, previously exposed to compounds A, A-7 and B (10 nM, 48 h) with BrdU (Bromodeoxyuridine, 10 μM added 24 hrs before the end of the treatment) and posterior immunostaining to detect BrdU positive cells. This is a well established method to determine cell division and proliferation by incorporation of the Uridine analogue. After counting BrdU positive oligodendrocytes versus total cells in three independent experiments we observed a slight tendency of increased proliferation in cells treated with compounds A, A-7 or B. However, there were not statistically significant differences between treated and control cells (control: 26.3±3.2; compound A: 29.2±4.0; compound A-7: 28,9±2.1; compound B: 27.6±6.7; Values are represented as Average±SEM).

### Induction of Myelin Regeneration by Small Molecules in Lysolecithin-Demyelinated Cerebellar Explants

Acute demyelination of cerebellum tissue explants has been previously used as a reliable methodology to study myelin formation and regeneration Taking advantage of our Pip-EGFP mouse, which allowed for the accurate tracking of oligodendrocyte cell behavior during myelination/demyelination/remyelination and the cerebellar explants methodology, we analyzed the effect of compounds A, A-7 and B or oligodendrocyte cells and myelin regeneration after demyeliantion of the cerebellar explants with lysolecithin. Cerebellum from postnatal day 7 mice pups (P7) was sectioned and maintained in culture (see methods). After only 4 days in vitro (div), numerous Plp-EGFP cells were observed distributed in the tissue explants, particularly concentrated at the cerebellar white matter and extending to the Purkinge layer. After 10 div the amount of Plp-EGFP cells increased significantly and the formation of myelin observed as axonal wrapping was easily detected by immunohistochemistry for MBP and neuronal filament (Data not shown). At this tune point (10 div), the explants were treated with lysolecithin (0.5 mg/ml, 17 hrs), which causes demyelination without affecting tissue structure or neuronal cell survival as judged for the integrity of dendrites and neuronal cell bodies observed after the treatment. Immediately after refreshing the tissue culture media, the demyelinated explants were treated with 0.1, 1 and 10 μM concentration of the small molecules A, A-7 and B. Media and treatment were renewed every 48 hrs thereafter for 10 additional days. Total time in culture was 20 days and 17 hrs. The tissue integrity was unaffected by the long-term culture as judged by the appearance of neuronal and glial cell bodies and their distribution in Purkinge layer and white matter areas.

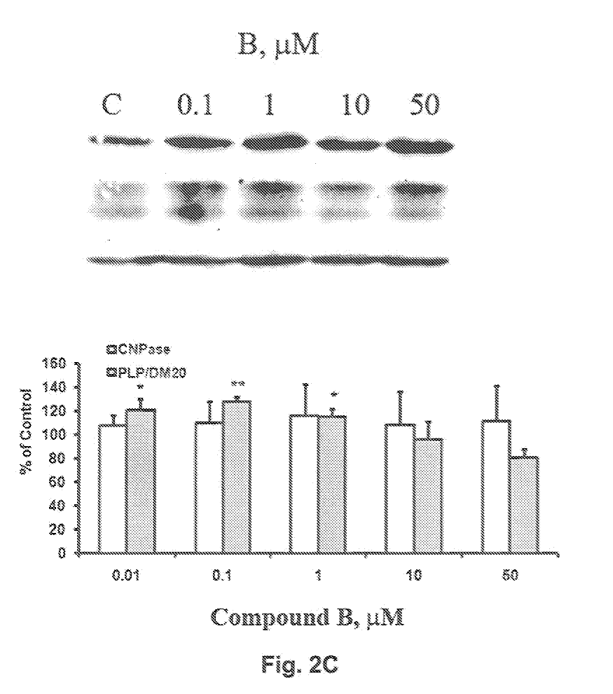

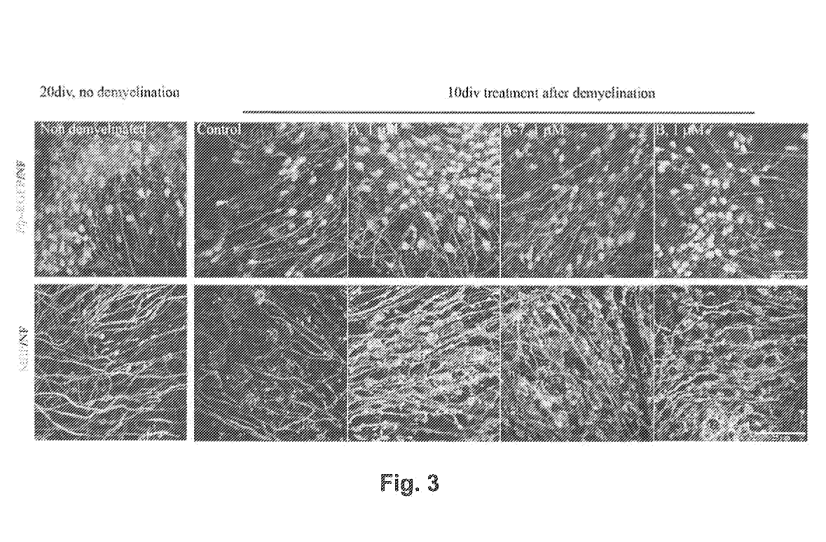

After this time in vitro, in explains not exposed to lysolecithin we observed abundant Plp-EGFP cells covering the entire tissue (FIG. 3, most left column, top panel) and showing a remarkable myelin formation and axonal wrapping as determined by MBP versus neuronal filament immunostaining (FIG. 3, most left column, bottom panel). In the explants exposed to lysolecithin and treated with vehicle, Plp-EGFP cell recovery was detectable as the oligodendrocyte progenitors were repopulating the demyelinated tissue (FIG. 3, Control column, top panel). However, myelin recovery was scarce and most neuronal axons were found nude (FIG. 3, Control column, bottom panel). Conversely, treatment of demyelinated explants with compounds A, A-7 and B (1 μM) resulted in a remarkable oligodendrocyte cell recovery which corresponded with a dramatic increase of myelin surrounding axonal extensions. The myelin regeneration effect induced by the selected small molecules, was detectable at the lowest concentration of compounds (0.1 μM) being at 1 μM that the most remarkable differences were found (FIG. 3). The relevant results obtained from these experiments using a tissue microenvironment assay suggested that our small molecules were indeed impacting oligodendrocyte cell and myelin regeneration and encouraged the subsequent studies implementing in viva approaches.

### Signalling Pathways Impacted by Small Mole Inducing Oligodendrocyte Precursor Differentiation

Oligodendrocyte precursor differentiation requires the activation of a complex, highly controlled machinery that signals cells to stop migrating/proliferating and start extending processes and producing myelin components. A number of inducers and intracellular mediators of this process have been identified in vitro and in viva. In recent studies, we demonstrated that protein kinase B, also known as Akt, induces oligodendrocyte differentiation and hypermyelination in the CNS. Therefore, we studied whether our selected small molecules induced Akt signaling in oligodendrocyte precursors. We analyzed Akt activity in cell extracts of oligodendrocyte precursors treated with selected compounds (10 μM, 48 h), as determined by phosphorylation of Akt substrate GSKα/β. Along with compounds A and B we also included in this experiment the small molecules C12H11FNOSBr—H (F), C15H17C1N2O3 (D) and C12H8F2N2O4S (C) which also showed induction of oligodendrocyte precursor maturation. Akt phosphorylation was clearly detected in response to compounds A, B and F.

Akt signaling has been extensively studied, and activation of phosphatidyl-inositol-3-kinase (PI3K) is generally accepted as a canonical upstream Akt activator. Downstream targets of Akt have also been identified and those include GSK, mTOR, p70SK, ASK amongst many others. In order to determine the relevance of P13R, signaling in the phosphorylation of Akt induced by compounds A and B and possible downstream targets of phosphorylated Akt, we pre-incubated the cells with inhibitors of PI3K (wortmannin, 0.1 μM), mTOR (rapamycin 0.1 μM) or a direct Akt ½ inhibitor (trifluoroacetate salt hydrate, 1 μM), all added 1 hr before the treatment with compounds A or B.

Cells were immunostained for MBP and after 48 h treatment with compound A or B the cells underwent morphology changes indicative of cell differentiation that included the generation of multiple cell processes and development of membranous structures. Inhibition of PI3K did not block oligodendrocyte precursor maturation induced by either compound. Akt inhibitor on the other hand, completely blocked the differentiation induced by both compounds indicating a major role of this signaling protein in oligodendrocyte precursor maturation induced by our selected small molecules. Inhibition of the downstream target of Akt, mTOR, also showed a differential effect on the maturation induced by the two analyzed compounds: while the effect of compound A was not modified, the oligodendrocyte precursor maturation induced by compound B was totally avoided.

### Study of the Effect of Small Molecules on Oligodendrocyte Precursor in Zebrafish In Vivo

The zebrafish has become an ideal model system to study not only early vertebrate development, but also numerous human diseases. The advantages of using zebrafish as oligodendrocyte development and myelination model are 1) the nervous system development in zebrafish is well characterized; 2) development occurs in a very short time course; 3) the zebrafish nervous system has fewer cells than those of mammals; and 4) the embryo is transparent so that early stages of development can be observed directly in the live organism. The transparent nature of embryos, together with the power of the transgenic technology to produce fluorescent zebrafish lines, makes it possible to visualize real-time oligodendrocyte precursor development, differentiation and myelination, as well as dysmyelination or demyelination, and to study the genetics of myelination and dysmyelination. We developed a transgenic fish using the same promoter cassette that drives EGFP expression in mammalian oligodendrocyte lineage cells. This is an excellent developmental model that facilitates the visual analysis of oligodendrocyte maturation and myelin formation in the vertebrate nervous system. We analyzed the effect of some of the primary hit compounds, which we had identified in our major small molecule screen of mouse cells in our zebrafish model as a reliable in vivo approach.

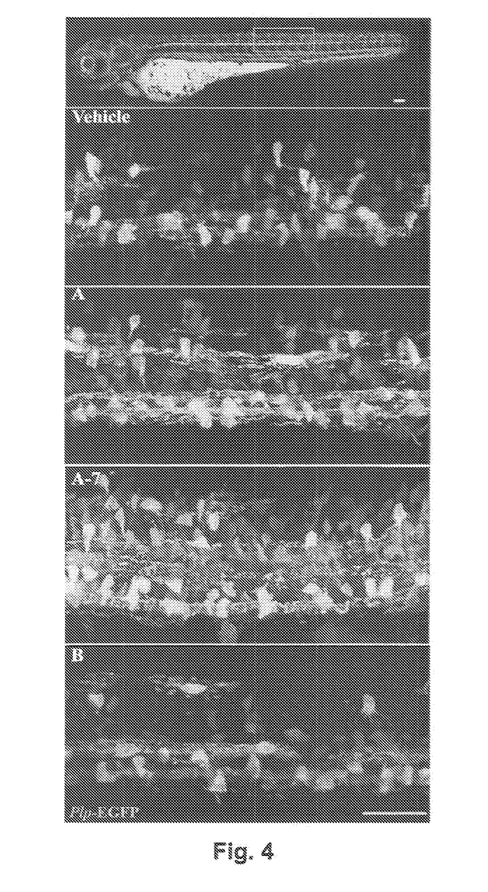

Embryos from transgenic-Plp-EGFP (tg[Plp:EGFP]) zebrafish were treated starting at 24 hours post fertilization (hpf) with compounds A, A-7 or B (1, 10 and 100 nM) or vehicle (DMSO). At 72 hpf effective treatments were analyzed by confocal microscopy after fixation and EGFP immunostaining of selected positive compounds. We observed a clear increase in the number of oligodendrocyte progenitor cells and fluorescence intensity in embryos exposed to compound A and A-7 (FIG. 4). These results demonstrate that these compounds are able to impact oligodendrocyte behavior in an in vivo model and in different species, as well as corroborate the specific effect of the selected small molecules on oligodendrocyte maturation.

### Effect of Selected Small Molecules on Remyelination After Cuprizone-Mediated Demyelination in Mice

Demyelination in mouse brain induced by the copper chelator cuprizone administered in the diet is a well established animal model to study demyelination and subsequent remyelination after cuprizone withdrawal. The effect of cuprizone is evident in several brain areas being the demyeliantion of the corpus callosum the most reliable and consistent. In this study, we analyzed the effect of compounds A, A-7 and B on remyelination of six week cuprizone-fed C57B1/6J mice. Compounds were administered i.p. at the end of the sixth week (5-100 mg/kg) while the mice were still maintained on the cuprizone diet for three more days. At that point, a second injection of compounds was performed and then the mice were returned to regular chow for two more days to allow the natural remyelination process to take place. Mice were then sacrificed and the brain tissue prepared for immunohistochemistry.

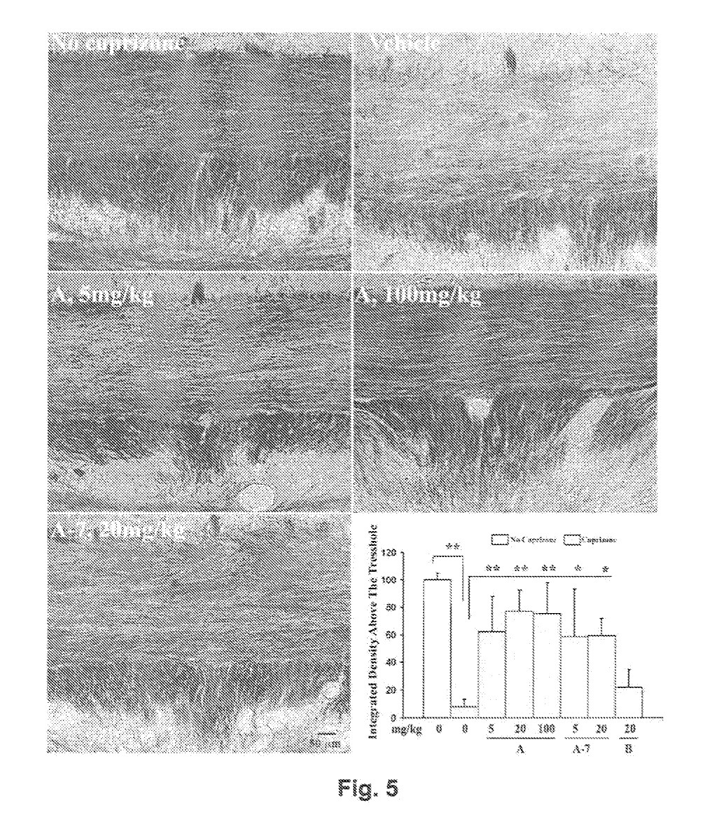

Black and Gold staining for total myelin in vehicle-injected, cuprizone-treated mice showed marked demyelination relative to control mice on normal feed. Demyelination was detected in the corpus callosum extending to the formix, as pale areas surrounded by dark black myelinated areas (FIG. 5, top row). On the other had cuprizone-treated mice that were injected with compound A and A-7 showed enhanced remyelination relative to vehicle-injected cuprizone-treated mice. Remyelination in response to these compounds was determined as areas with increased. Black and Gold stain, clearly observed on the corpus callosum and the formix of injected animals indicating enhanced remyelination (FIG. 5). The enhanced remyelination effect induced by compound A was highly significant at the lowest administered dose (5 mg/kg) reaching 80% myelin levels of the non-cuprizone-fed animals as the dose increased to 100 mg/kg. Compound A-7 also induced a highly significant remyelination effect which appeared slightly lower that that observed for compound A at the same doses. We did not detect a measurable induction of myelin recovery in response to compound B (FIG. 5).

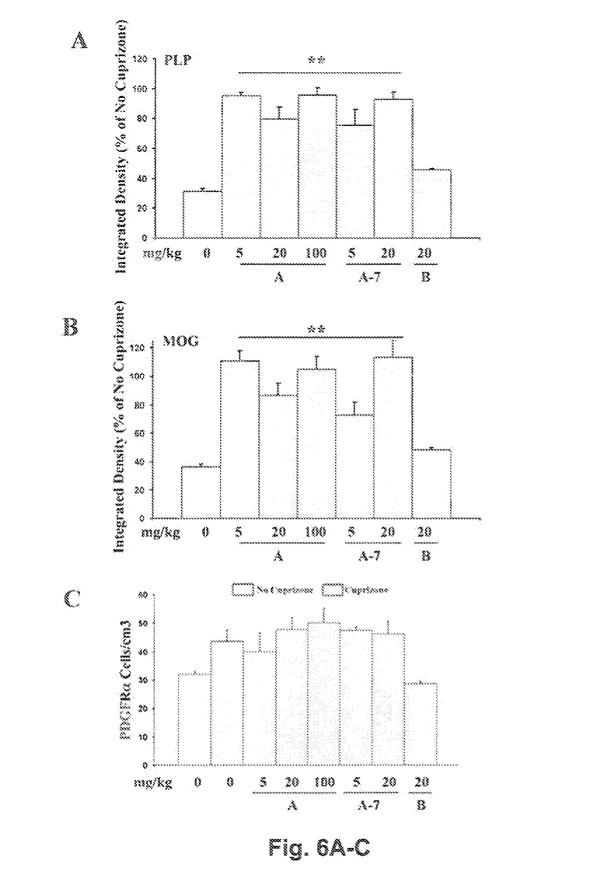

Immunostaining for the myelin proteins PLP/DM20, MBP and MOG further confirmed the effect of compounds A and A-7 on the myelin regeneration in cuprizone-demyelinated mice (FIG. 6). Densitometry lysis of brain tissue sections stained for PLP/DM20 and MOG using the Odyssey infrared scanner, indicated that in the corpus callosum there was a 65% loss of myelin protein stain in vehicle-injected cuprizone-treated mice, relative to control mice in normal diet. Myelin protein levels increased in mice injected with compound A, approaching control, non-cuprizone-treated levels in the corpus callosum (FIG. 6). Once again, while we did not detect any relevant effect in response to compound B, the remyelination enhancement caused by compounds A and A-7 was already observed at low dose reaching 80-90% the non cuprizone-fed Myelin protein levels in animals injected with 5-20 mg/kg solutions of these two chemicals.

The outstanding induction of remyelination by our small molecules in an w vivo demyeliantion model required the ability of the compounds to cross the blood brain barrier and once in the brain, specifically activate the endogenous oligodendrocyte progenitors to mobilize to the demyelinated areas, proliferate, differentiate, target axons and generate new myelin. To establish if the selected compounds actually complied with these requirements, we first determine if the enhanced myelination was the result of increased proliferation of oligodendrocyte progenitors in the demyelinated areas. To this end, we immunostained tissue sections from all the conditions with the oligodendrocyte progenitor cell marker plateled-derived growth factor receptor alpha (PDGFRα) and NG2-proteoglycan it shown) and determined cell numbers in the Irma of interest. NG2⁺ cells were clearly evident and relatively easily detected by immunostaining. However, the highlighted cell processes and extensions rendered the counting process unviable. PDGFRα⁺ cells, on the other hand, showed a well defined immunostaining of the cell body which facilitated the counting. Therefore, we counted PDGFRα⁺ cells in 25 μm thick confocal microscopy Z-series enclosed in the corpus callosum area (FIG. 6, C). We detected a significant increase of these cells in the cuprizone-fed mice as compared with non-cuprizone diet. However, no significant differences were observed between the vehicle injected mice and the compound injected animals at this time point (FIG. 6, E).

The next analysis we performed, was aimed to assess whether the injected compounds were able to cross the blood brain barrier, This would definitely indicate that the observed effects on myelin regeneration were indeed induced by the impact of the injected compounds on oligodendrocyte progenitor differentiation. 4 mice per condition were injected i.p. (100 mg/kg), following the same experimental paradigm performed for the tissue analysis (two doses m a 5 days time). The presence of compounds A and A-7 in the blood plasma and brain tissue was analyzed by mass spectrometry. Most remarkably, the two compounds A and A-7 were present in both blood plasma and brain tissue well above the detection limit of the mass spectrometry standard curve, made with a solution of pure compound dissolved in DMSO (See methods). As observed in Table 4, blood plasma and brain levels of compound A were high relative to the method's limit of detection (LOD: 1.5 and 0.5 respectively), averaging 131.3 in blood plasma and 9.2 nM in brain. For compound A-7, although lower levels were detected as compared to compound A, those levels were much higher than the LOD, which was 1.5 nM for both blood plasma and brain. Compound A-7 values averaged 9.2 and 13.5 nM respectively (Table 5).

TABLE 5
Biological sample concentration (nM)

| Plasma | | | | | | Brain | | | | |
|---|---|---|---|---|---|---|---|---|---|---|
| Sample No. | LOD | 1 | 2 | 3 | 4 | LOD | 1 | 2 | 3 | 4 |
| Vehicle | UD | LOD | LOD | LOD | LOD | UD | LOD | LOD | LOD | LOD |
| A | 1.5 | 90 | 149 | 129 | 157 | 0.5 | 4.6 | 14.7 | 13.2 | 4.3 |
| A-7 | 1.5 | 4.2 | 5.3 L | OD | 3.6 | 1.5 | 16.6 | 16.8 | 4.21 | 6.2 |

The identified compounds that induced clear, measurable effects in in vitro models were also highly effective n two different in vivo contexts. The two in vivo models we used to test our small molecules provided valuable information on the effect of the compounds during oligodendrocyte development in the spinal cord of zebrafish and on myelin regeneration on an adult rodent demyeliantion model. Our observations indicate that the selected compounds acted specifically on oligodendrocyte precursor cells inducing a differentiation effect that resulted in an important enhancement of myelin formation both during development which and during remyelination. Importantly, compounds A and A-7 were found in blood plasma and brain tissue after two I.P. doses in a five day-term, being the last injection administered at least 24 hrs before the recollection of the specimens. This finding indicates that the compounds effectively crossed the blood brain barrier and specifically acted on resident oligodendrocyte precursors, inducing differentiation of these cells and finally enhancing myelin formation. A more detailed pharmacokinetic analysis is necessary to determine pm-clinical characteristics of these small molecules, such as tissue distribution, cytotoxicity in in vivo models and elimination vias.

### EXAMPLE 2

### Synthesis of Compound A-7

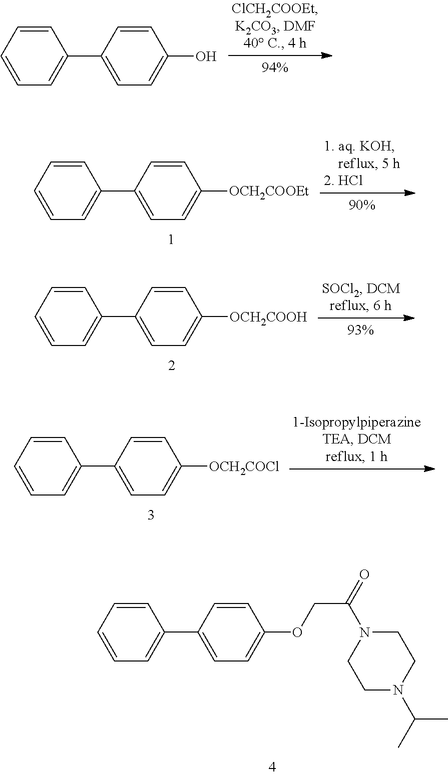

Ethyl (biphenyl-4-yloxy)acetate (1). A mixture of biphenyl-4-ol (17 g, 0.1 mol), K₂CO₃ (17.9 g, 0.13 mol), ethyl chloroacetate (14.7 g, 0.12 mol) and DMF (70 mL) was stirred for 4 hr at 80° C. cooled, and poured under vigorous stirring into water (300 mL). The resulting precipitate was filtered off, washed thoroughly with water and crystallized from ethanol to yield 24 g (94%) of ester 1 as a colorless solid.

(Biphenyl-4-yloxy)acetic acid (2). A mixture of ester 1 (12.8 g, 50.0 mmol) and KOH (8.4 g, 150 mmol) in water (100 mL) was stirred under reflux for 5 hr and then cooled. To the resulting, suspension of potassium salt was added cone. HCl (17 mL), and the mixture was refluxed for another 1 hr, cooled. The precipitate was filtered off, washed with water and dried at 100° C. to give 10.0 g (90%) of acid 2 as a colorless solid.

(Biphenyl-4-yloxy)acetyl chloride (3). A mixture of acid 2 (2.3 g, 10 mmol), SOCl₂ (2.5 g, 21 mmol) and dichloromethane (50 mL) was stirred under reflux for 6 hr and then cooled. Most of the solvent was removed in vacua, and the acyl chloride 3 was precipitated by addition of hexane (50 mL), filtered off, washed with hexane and dried th vacuo to give 2.3 g (93%) of acid chloride 3.

1-(Biphenyl-4-yloxy)acetyl-4-isopropylpiperazine (4). To a solution of 1-isopropylpiperazine (2.56 g, 20 mmol) and triethylamine (2.02 g, 20 mmol) in dichloromethane (40 mL) was added a solution of acid chloride 3 (1.25 g, 5.0 mmol). The mixture was stirred under reflux for 1 hr, cooled, washed twice with 5% KOH, then with water and evaporated in vacuo to dryness. The residue was crystallized from hexane to yield 1.1 g (65%) of the desired amide 4 as a colorless solid.

All publications and patents mentioned in the above specification are herein incorporated by reference.

1. A method of promoting oligodendrocyte precursor cell differentiation comprising administering to one or more oligodendrocyte precursor cells an effective amount of a compound having the following general structure: 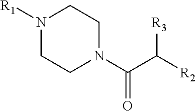
wherein R₁, R₂, and R₃ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, salts thereof, and combinations thereof.

2. The method of claim 1 the compound being selected from the group consisting of: 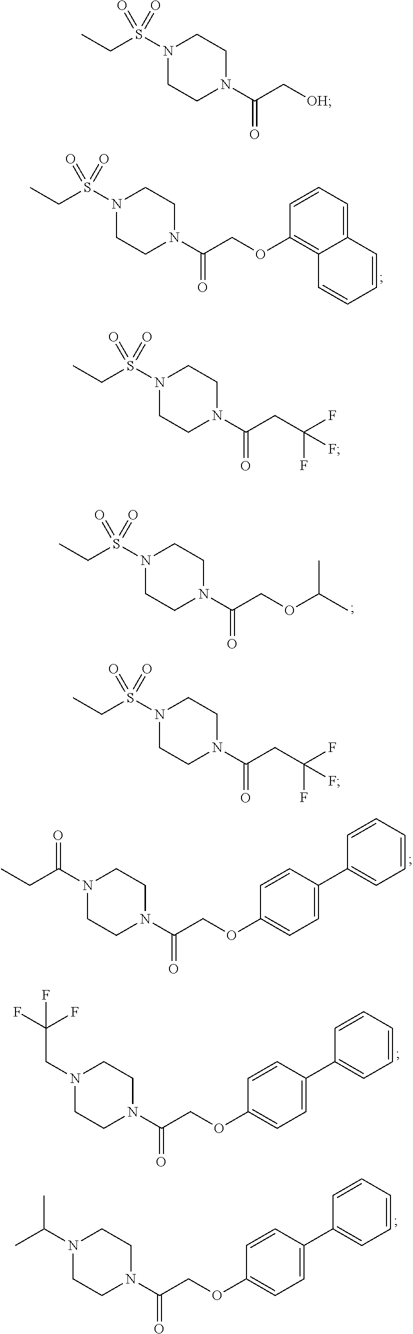 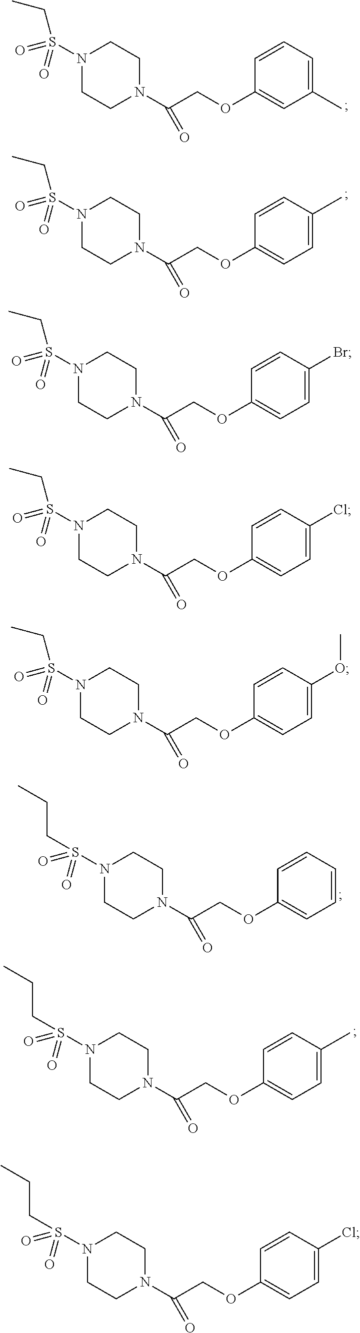 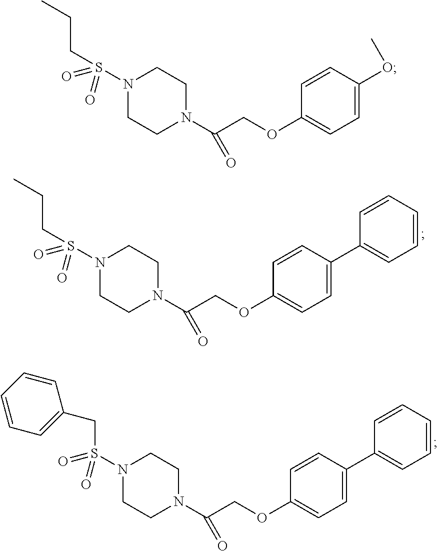
and salts thereof.

3-8. (canceled)

9. A method of treating a neurodegenerative disease in a subject comprising administering to the subject a therapeutically effective amount of a compound having the following general structure: 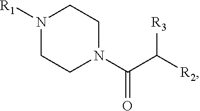
wherein R₁, R₂, R₃ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, salts thereof, and combinations thereof.

10. The method of claim 9, wherein the subject is a mammal.

11. (canceled)

12. The method of claim 9, wherein the subject is suspected of having a demyelinating disease.

13. The method of claim 12, the demyelinating disease comprising multiple sclerosis.

14. A method of treating multiple sclerosis in a subject comprising administering to the subject a therapeutically effective amount of a compound having the following general structure: 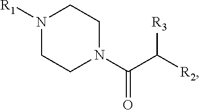
wherein R₁, R₂, R₃ each independently represent substituents selected from the group consisting of hydrogen, an alkyl, an alkenyl, an alkynyl, an aryl, an alkaryl, an aralkyl, a halo, hydroxyl, an alkoxy, an alkenyloxy, an alkynyloxy, an aryloxy, acyloxy, an alkoxycarbonyl, an aryloxycarbonyl, a halocarbonyl, an alkylcarbonato, an arylcarbonato, a carboxy, a carboxylato, a carbamoyl, an amino, a substituted amino, an alkylamido, an arylamido, an imino, an alkylimino, an arylimino, a nitro, a nitroso, salts thereof, and combinations thereof.

15. The method of claim 14, wherein the subject is a mammal.

16. The method of claim 15, wherein the mammal is a human.

17-25. (canceled)

26. The method of claim 1, wherein the compound is administered to oligodendrocytes of a subject.

27. The method of claim 26, wherein the subject is a mammal.

28. The method of claim 27, wherein the mammal is a human.

29. The method of claim 26, wherein the subject is suspected of having a neurodegenerative disease.

30. The method of claim 26, wherein the subject is suspected of having a demyelinating disease.

31. The method of claim 27, the demyelinating disease comprising multiple sclerosis.

32. The method of claim 1, the compound comprising the following structure:

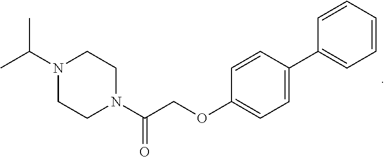

33. The method of claim 9 the compound being selected from the group consisting of: 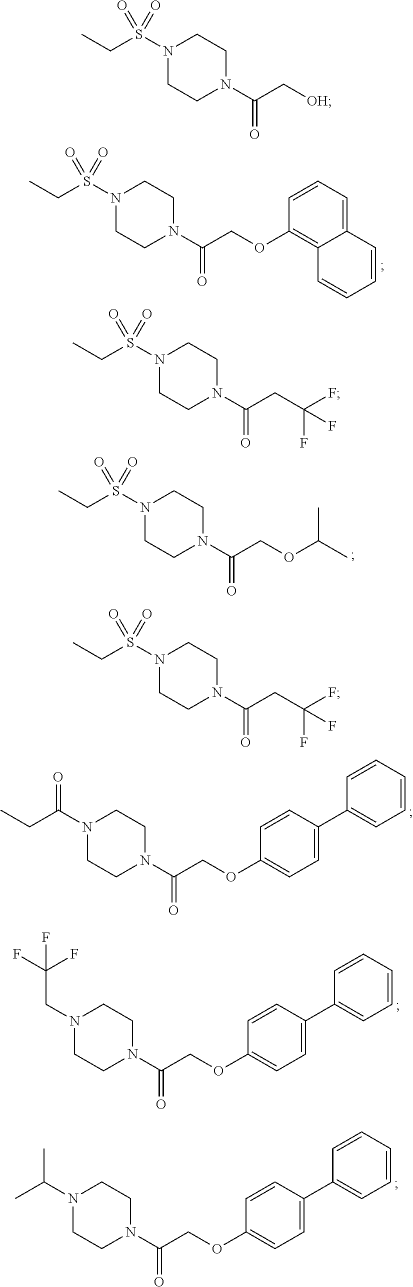 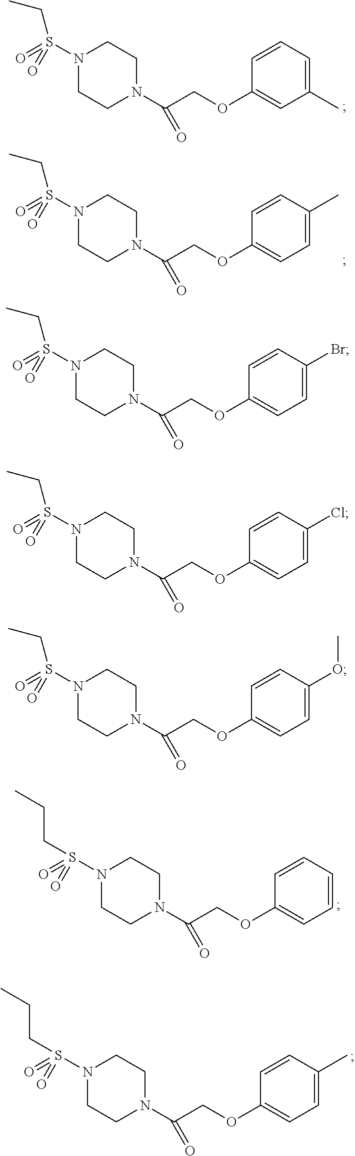 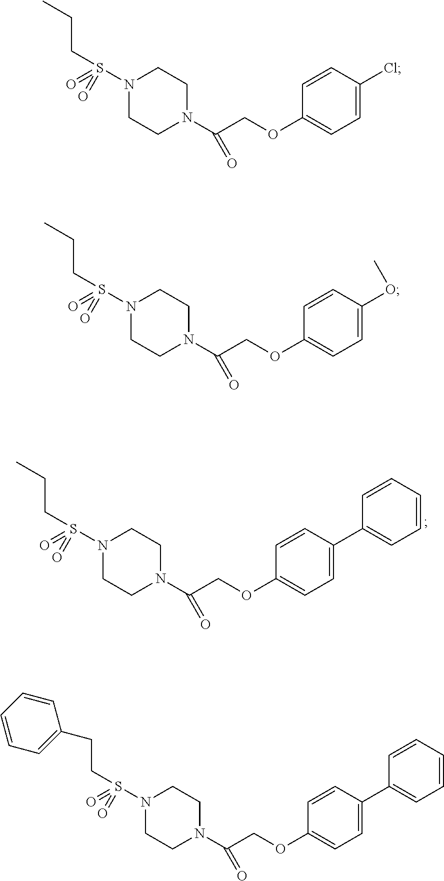
and salts thereof.

34. The method of claim 33, the compound comprising the following structure:

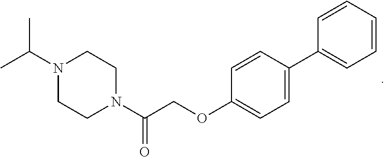

35. The method of claim 14 the compound being selected from the group consisting of: 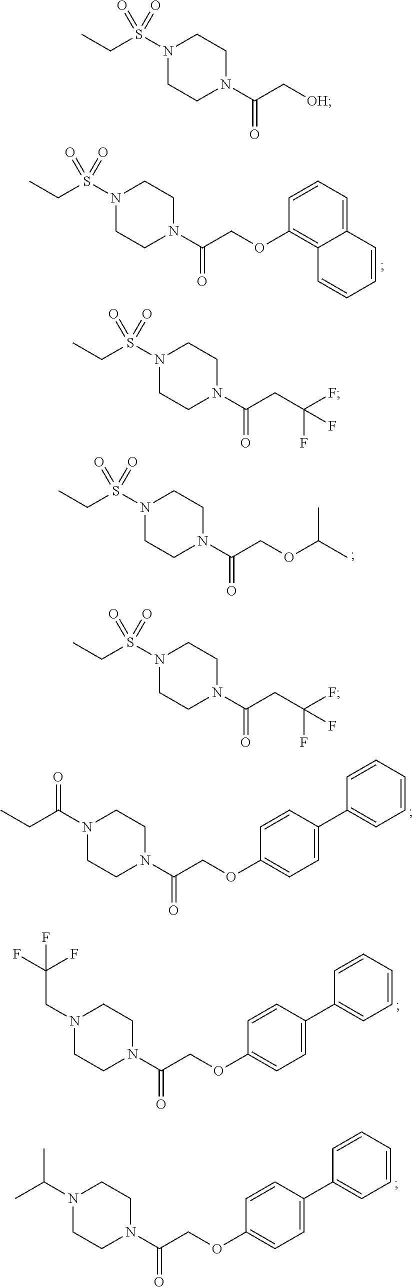 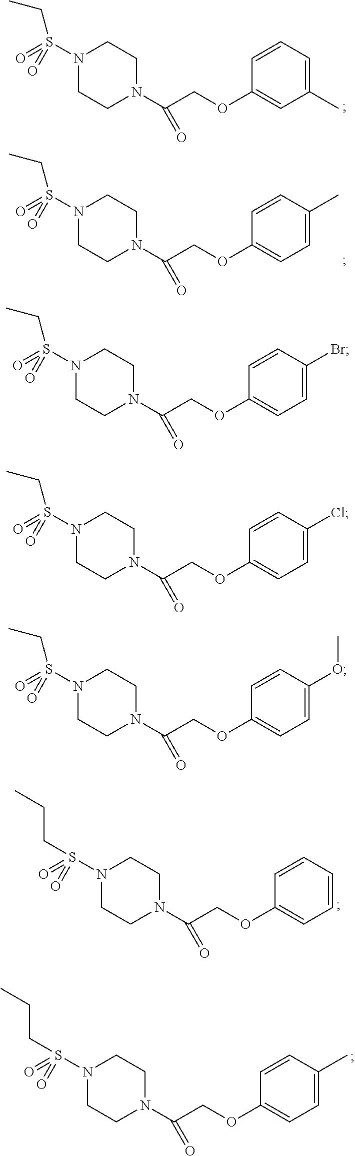 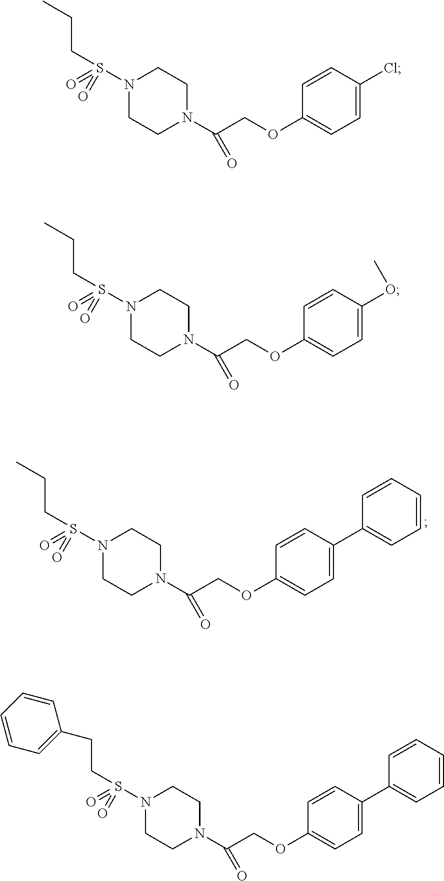
and salts thereof.

36. The method of claim 35, the compound comprising the following structure: [chemical structure]
